(19) United States
(12) Patent Application Publication
(10) Pub. No.: US 2018/0273700 A1
(43) Pub. Date: Sep. 27, 2018

(54) GRAPHENE-BIOPOLYMER COMPOSITE MATERIALS AND METHODS OF MAKING THEREOF

(71) Applicants: THE BOARD OF TRUSTEES OF THE UNIVERSITY OF ALABAMA, Tuscaloosa, AL (US); 525 Solutions, Inc., Tuscaloosa, AL (US)

(72) Inventors: Robin D. Rogers, Tuscaloosa, AL (US); Oleksandra Zavgorodnya, Tuscaloosa, AL (US); Julia L. Shamshina, Tuscaloosa, AL (US); Gabriela Gurau, Tuscaloosa, AL (US)

(21) Appl. No.: 15/936,056

(22) Filed: Mar. 26, 2018

Related U.S. Application Data
(60) Provisional application No. 62/476,013, filed on Mar. 24, 2017.

(51) Int. Cl. C08J 3/20; C08B 37/08; C08K 3/04; C09D 105/08; B01J 20/24; B01J 20/20; B01J 20/30; A61L 27/20; A61L 27/08; D01D 5/00; D01F 1/10; D01F 9/00; D01D 1/02

(52) U.S. Cl. CPC C08J 3/203; C08K 2201/006; C08K 3/042; C09D 105/08; B01J 20/24; B01J 20/205; B01J 20/305; B01J 20/3007; A61L 27/20; A61L 27/08; D01D 5/0046; D01F 1/10; D01F 9/00; D01D 1/02; C08K 2201/001; C08K 2201/011; C08J 2305/08; C08B 37/003

(57) ABSTRACT

Methods for making graphene-biopolymer composite materials are described. The methods can comprise contacting an ionic liquid with a biopolymer and graphene, thereby forming a mixture; contacting the mixture with a non-solvent, thereby forming the graphene-biopolymer composite material in the non-solvent; and collecting the graphene-biopolymer composite material from the non-solvent.

### CROSS-REFERENCE TO RELATED APPLICATIONS

This application claims the benefit of priority to U.S. Provisional Application 62/476,013, filed Mar. 24, 2017, which is hereby incorporated herein by reference in its entirety.

### BACKGROUND

Graphene is a two-dimensional monolayer of carbon atoms that possesses remarkable mechanical, electrical and thermal properties. In addition, graphene has a large surface area and can be a safer analog to carbon nanotubes, which makes graphene an attractive candidate for biomedical applications, conductive textile coatings, optical elements, battery electrode materials, etc. Among different graphene-based materials, polymer-graphene nanocomposites have gained significant attention due, at least in part, to their combination of the properties of graphene, such as thermal and electrical conductivity, thermal stability, mechanical, optical properties, and the flexibility of polymers, including processability into a variety of material shapes.

Accordingly, polymer-graphene nanocomposites comprising graphene dispersed in a polymer matrix have been the subject of numerous developments. These graphene-containing materials can be made by a variety of techniques such as melt-blending, electrospinning, doping, chemical vapor deposition and self-assembly to yield materials of different shapes and sizes including nanofibers, membranes, and papers. Yet, conventional polymer nanocomposites suffer from limitations related to 1) type of polymers, i.e. mostly synthetic polymeric materials are prepared, and 2) uneven dispersion of the graphene that can diminish performance attributes of the resultant material. Several other problems with conventional composite materials include use of expensive nanotubes rather than graphene, material preparation methods that are impractical for large-scale commercial production and processing difficulties. Specific interest lies in the area of polymer composites in which the graphene particles are uniformly dispersed in the polymer matrix, therefore a need for making such composites exists. The methods described herein address these and other needs.

### SUMMARY

In accordance with the purposes of the disclosed systems and methods, as embodied and broadly described herein, the disclosed subject matter relates to methods of making a graphene-biopolymer composite material.

Additional advantages of the disclosed process will be set forth in part in the description which follows, and in part will be obvious from the description, or can be learned by practice of the disclosed process. The advantages of the disclosed process will be realized and attained by means of the elements and combinations particularly pointed out in the appended claims. It is to be understood that both the foregoing general description and the following detailed description are exemplary and explanatory only and are not restrictive of the disclosed process, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification illustrate several aspects described below.

FIG. 1 is a typical SEM image of dry nanopowder of graphene grade AO-2.

FIG. 2 is a typical SEM image of dry nanopowder of graphene grade AO-3.

FIG. 3 is a typical SEM image of dry nanopowder of graphene grade AO-4.

FIG. 4 is a typical SEM image of dry nanopowder of graphene grade C1.

FIG. 5 is a schematic diagram of electrospinning apparatus.

FIG. 6 is a photograph of electrospinning apparatus.

FIG. 7 is the Powder X-Ray Diffraction (PXRD) data for shrimp shell chitin/graphene electrospun composites with 0.0012 wt % of graphene (AO-4).

FIG. 8 is an optical microscopy image of electrospun shrimp shell chitin/graphene (AO-4) composite with a 0.0012 wt % graphene concentration.

FIG. 9 is an optical microscopy image of electrospun shrimp shell chitin/graphene (AO-4) composite with 0.0012 wt % graphene concentration.

FIG. 10 is an optical microscopy image of electrospun shrimp shell chitin/graphene (AO-4) composite with 0.01 wt % graphene concentration.

FIG. 11 is an optical microscopy image of electrospun shrimp shell chitin/graphene (AO-4) composite with 0.01 wt % graphene concentration.

FIG. 12 is an atomic force microscopy (AFM) image of composite shrimp shell chitin/graphene with 0.0012 wt % of graphene. Scan size: 2×2 μm².

FIG. 13 is an AFM image of composite shrimp shell chitin/graphene with 0.0012 wt % of graphene. Scan size: 1×1 μm².

FIG. 14 is an AFM image of composite shrimp shell chitin/graphene with 0.0054 wt % of graphene. Scan size 1×1 μm².

FIG. 15 is an SEM image of electrospun shrimp shell chitin/graphene (0.0054 wt %) composite mats.

FIG. 16 is an SEM image of electrospun shrimp shell chitin/graphene (0.0054 wt %) composite mats.

FIG. 17 is a photograph of an electrospun regenerated chitin/graphene (AO-2) nanomat on the water surface.

FIG. 18 is a photograph of an electrospun regenerated chitin/graphene (AO-2) nanomat on the water surface.

FIG. 19 is a graph of the PXRD data for AO-2 graphene nanopowder and regenerated chitin/AO-2 composite mats (graphene concentration 0.0054 wt %).

FIG. 20 is an optical microscopy image of regenerated chitin/graphene (AO-2, 0.0054 wt %) composite mats (magnification 40×).

FIG. 21 is an optical microscopy image of regenerated chitin/graphene (AO-2, 0.0054 wt %) composite mats (magnification 40×).

FIG. 22 is an optical microscopy image of regenerated chitin/graphene (AO-4, 0.0054 wt %) composite mats (magnification 40×).

FIG. 23 is an optical microscopy image of regenerated chitin/graphene (AO-4, 0.0054 wt %) composite mats (magnification 40×).

FIG. 24 is a photograph demonstrating electrospinning of composite solutions on solid support.

FIG. 25 is a schematic representation of the dry-wet spinning technique.

FIG. 26 is an optical microscope images of chitin-graphene fibers with graphene load of 0.005 wt % at 4× magnification.

FIG. 27 is an optical microscope images of chitin-graphene fibers with graphene load of 0.005 wt % at 10× magnification.

FIG. 28 is an O=optical microscope images of chitin-graphene fibers with graphene load of 0.005 wt % at 40× magnification.

FIG. 29 is an optical microscope images of chitin-graphene fibers with graphene load of 0.005 wt % at 40× magnification.

FIG. 30 is an optical microscope images of chitin-graphene fibers with graphene load of 0.005 wt % at 100× magnification.

FIG. 31 is an optical microscope image of chitin-graphene fibers with graphene load of 0.01 wt % at 4× magnification.

FIG. 32 is an optical microscope image of chitin-graphene fibers with graphene load of 0.01 wt % at 10× magnification.

FIG. 33 is an optical microscope image of chitin-graphene fibers with graphene load of 0.01 wt % at 40× magnification.

FIG. 34 is an optical microscope image of chitin-graphene fibers with graphene load of 0.01 wt % at 40× magnification.

FIG. 35 is an optical microscope image of chitin-graphene fibers with graphene load of 0.01 wt % at 100× magnification.

FIG. 36 shows the stress-strain curves obtained for chitin and chitin-graphene fibers.

FIG. 37 is an optical image of chitin-graphene films with 0.005 wt % of graphene.

FIG. 38 is a photograph of chitin-graphene films with 0.005 wt % of graphene.

FIG. 39 is a photograph of a dry chitin film.

FIG. 40 is a photograph of a dry chitin-graphene loaded film.

FIG. 41 is a photograph of a dry chitin-graphene loaded film.

FIG. 42 is a SEM image at 2000× magnification for a neat chitin film showing the surface morphology.

FIG. 43 is a SEM image at 2000× magnification for an 80 wt % graphene/chitin composite film showing the surface morphology.

FIG. 44 shows the thermogravimetric analysis (TGA) of neat chitin, chitin powder, graphene powder and graphene/chitin composite films (with the mass of the composite film normalized to the mass of chitin).

FIG. 45 shows the results of the tensile tests of the neat chitin film and the graphene/chitin composite film.

### DETAILED DESCRIPTION

The materials, compounds, compositions, articles, and methods described herein can be understood more readily by reference to the following detailed description of specific aspects of the disclosed subject matter and the Examples and Figures included therein.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an ionic liquid” includes mixtures of two or more such ionic liquids, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included. The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., Zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation acetylation, esterification, deesterification, hydrolysis, etc.

The term “anion” is a type of ion and is included within the meaning of the term “ion.” An “anion” is any molecule, portion of a molecule (e.g., Zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., Zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-Cis, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphtyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C1-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a short hand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula —C(O)O⁻.

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z′, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

The term “hydrogen bond” describes an attractive interaction between a hydrogen atom from a molecule or molecular fragment X—H in which X is more electronegative than H, and an atom or a group of atoms in the same or different molecule, in which there is evidence of bond formation. The hydrogen bond donor can be a cation and the hydrogen bond acceptor can be an anion.

The term “complex” describes a coordination complex, which is a structure comprised of a central atom or molecule that is weakly connected to one or more surrounding atoms or molecules, or describes chelate complex, which is a coordination complex with more than one bond.

References to “mim,” “Cₙ-mim,” and “bmim” are intended to refer to a methyl imidazolium compound, an alkyl (with n carbon atoms) methyl imidazolium compound, and a butyl methylimidazolium compound respectively.

As used herein, the term “chitosan” means deacetylated chitin (at least 50% deacetylated) or any other form of chemically modified chitin.

Reference will now be made in detail to specific aspects of the disclosed materials, compounds, compositions, formulations, articles, and methods, examples of which are illustrated in the accompanying Examples and Figures.

### Methods

Disclosed herein are methods of making graphene-biopolymer composite materials, the methods comprising contacting an ionic liquid with a biopolymer and graphene, thereby forming a mixture.

The term “ionic liquid” has many definitions in the art, but is used herein to refer to salts (i.e., an ionic compound of cations and anions) that are liquid at a temperature of at or below 150° C. That is, at one or more temperature ranges or points at or below 150° C. the disclosed ionic liquid compositions are liquid; although, it is understood that they can be solids at other temperature ranges or points. See e.g., Wasserscheid and Keim, Angew Chem Int Ed Engl, 2000, 39:3772; and Wasserscheid, “Ionic Liquids in Synthesis,” 1″ Ed., Wiley-VCH, 2002.

In some examples, the ionic liquid can be a liquid at a temperature of 150° C. or less (e.g., 140° C. or less, 130° C. or less, 120° C. or less, 110° C. or less, 100° C. or less, 90° C. or less, 80° C. or less, 70° C. or less, 60° C. or less, 50° C. or less, 40° C. or less, 30° C. or less, 20° C. or less, 10° C. or less, 0° C. or less, −10° C. or less, −20° C. or less, or −30° C. or less). Further, in some examples the disclosed ionic liquids can be liquid over a range of temperatures. For example, the disclosed ionic liquids can be liquids over a range of 1° C. or more (e.g., 2° C. or more, 3° C. or more, 4° C. or more, 5° C. or more, 6° C. or more, 7° C. or more, 8° C. or more, 9° C. or more, 10° C. or more, 11° C. or more, 12° C. or more, 13° C. or more, 14° C. or more, 15° C. or more, 16° C. or more, 17° C. or more, 18° C. or more, 19° C. or more, or 20° C. or more). Such temperature ranges can begin and/or end at any of the temperature points disclosed above.

In further examples, the disclosed ionic liquids can be liquid at temperature from −30° C. to 150° C. (e.g., from −20° C. to 140° C., −10° C. to 130° C., from 0° C. to 120° C., from 10° C. to 110° C., from 20° C. to 100° C., from 30° C. to 90° C., from 40° C. to 80° C., from 50° C. to 70° C., from −30° C. to 50° C., from −30° C. to 90° C., from −30° C. to 110° C., from −30° C. to 130° C., from −30° C. to 150° C., from 30° C. to 90° C., from 30° C. to 110° C., from 30° C. to 130° C., from 30° C. to 150° C., from 0° C. to 100° C., from 0° C. to 70° C., or from 0° to 50° C.).

Further, exemplary properties of ionic liquids are high ionic range, non-volatility, non-flammability, high thermal stability, wide temperature for liquid phase, highly solvability, and non-coordinating. For a review of ionic liquids see, for example, Welton, Chem Rev., 99:2071-2083, 1999; and Carlin et al., Advances in Nonaqueous Chemistry, Mamantov et al. Eds., VCH Publishing, New York, 1994. These references are incorporated by reference herein in their entireties for their teachings of ionic liquids.

The term “liquid” describes the compositions that are generally in amorphous, non-crystalline, or semi-crystalline state. For example, while some structured association and packing of cations and anions can occur at the atomic level, an ionic liquid composition can have minor amounts of such ordered structures and are therefore not crystalline solids. The compositions can be fluid and free-flowing liquids or amorphous solids such as glasses or waxes at temperatures at or below 150° C.

The ionic liquids of the present disclosure can comprise an organic cation and an organic or inorganic anion. The organic cation is typically formed by alkylation of a neutral organic species capable of holding a positive charge when a suitable anion is present.

Further, the ionic liquid can be composed of at least two different ions, each of which can independently and simultaneously introduce a specific characteristic to the composition not easily obtainable with traditional dissolution and formulation techniques. Thus, by providing different ions and ion combinations, one can change the characteristics or properties of the disclosed compositions in a way not seen by simply preparing various crystalline salt forms.

Examples of characteristics that can be controlled in the disclosed compositions include, but are not limited to, melting, solubility control, rate of dissolution, and a biological activity or function. It is this multi-nature/functionality of the disclosed ionic liquid compositions which allows one to fine-tune or design in very specific desired material properties. For example, the ionic liquids of the present disclosure can comprise at least one cation and at least one anion.

The choice of the cation in the ionic liquid can be particularly relevant to the rate and level of graphene dissolution. While not wishing to be bound by theory, the primary mechanism of solvation of graphene by an ionic liquid is the cation's ability to interact with the π-electrons of graphene. Thus, it is believed that that the dissolution of chitin is enhanced by increasing the ability of the cation to interact with the π-electrons of graphene, for example by using an aromatic cation, such as an imidazolium cation. The interaction of the ionic liquids with the graphene can be influenced by the charge transfer between the component ions (Ghatee M H et al. J. Phys. Chem. C. 2011, 115, 5626-5636). The aromaticity of the cation in the ionic liquid can result in unique charge transfer interactions and enhanced π-interactions with graphene.

The organic cation of the ionic liquids disclosed herein can comprise a linear, branched, or cyclic heteroalkyl unit. The term “heteroalkyl” refers to a cation as disclosed herein comprising one or more heteroatoms chosen from nitrogen, oxygen, sulfur, boron, or phosphorous capable of forming a cation. The heteroatom can be a part of a ring formed with one or more other heteroatoms, for example, pyridinyl, imidazolinyl rings, that can have substituted or unsubstituted linear or branched alkyl units attached thereto. In addition, the cation can be a single heteroatom wherein a sufficient number of substituted or unsubstituted linear or branched alkyl units are attached to the heteroatom such that a cation is formed. For example, the cation [Cₙmim] where n is an integer of from 1 to 8 can be used. Preferably, ionic liquids with the cation [C₁₋₄ mim] can be used. A particularly useful ionic liquid is 1-ethyl-3-methyl-1H-imidazol-3-ium acetate, [C₂mim]OAc, having the formulae:

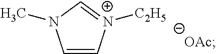

is an example of an ionic liquid comprising a cyclic heteroalkyl cation; a ring comprising 3 carbon atoms and 2 nitrogen atoms.

Other non-limiting examples of heterocyclic and heteroaryl units that can be alkylated to form cationic units include imidazole, pyrazoles, thiazoles, isothiazoles, azathiozoles, oxothiazoles, oxazines, oxazolines, oxazaboroles, dithiozoles, triazoles, selenozoles, oxahospholes, pyrroles, boroles, furans, thiphenes, phospholes, pentazoles, indoles, indolines, oxazoles, isothirazoles, tetrazoles, benzofurans, dibenzofurans, benzothiophenes, dibenzothoiphenes, thiadiazoles, pyrdines, pyrimidines, pyrazines, pyridazines, piperazines, piperidines, morpholines, pyrans, annolines, phthalazines, quinazolines, and quinoxalines.

The following are examples of heterocyclic units that are suitable for forming a cyclic heteroalkyl cation unit of the disclosed ionic liquids:

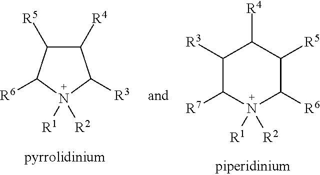

The following are further examples of heterocyclic units that are suitable for forming a cyclic heteroalkyl cation unit of the disclosed ionic liquids:

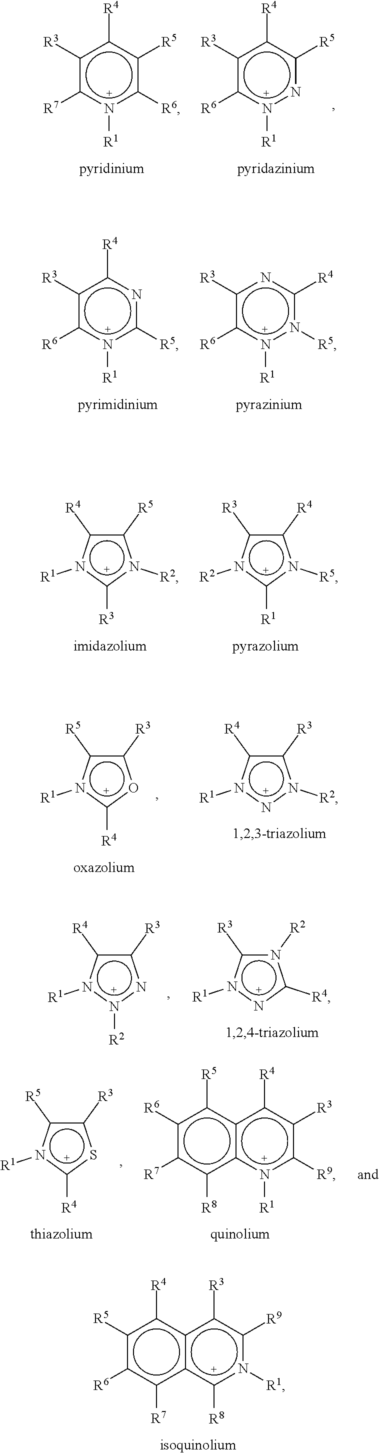

where each R¹ and R² is, independently, a substituted or unsubstituted linear, branched, or cyclic C₁-C₆ alkyl, or substituted or unsubstituted linear, branched, or cyclic C₁-C₆ alkoxy; each R³, R⁴, R⁵, R⁶, R⁷, R⁸, and R⁹ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl.

The following comprises yet another set of examples of heterocyclic units that are suitable for forming heterocyclic dication units of the disclosed ionic liquids and are referred to as such or as “geminal ionic liquids:” See Armstrong, D. W. et al., Structure and properties of high stability geminal dicationic ionic liquids, J. Amer. Chem. Soc. 2005; 127(2):593-604; and Rogers, R. D. et al., Mercury(II) partitioning from aqueous solutions with a new, hydrophobic ethylene-glycol functionalized bis-imidazolium ionic liquid, Green Chem. 2003; 5:129-135 included herein by reference in its entirety.

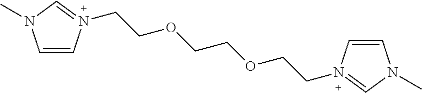

### 1,1′-[1,2-ethanediylbis(oxy-1,2-ethanediyl)]bis[3-methyl-1H-imidazolium-1-yl]

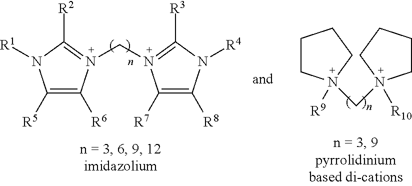

where R¹, R⁴, R⁹, and R¹⁰ comprise a substituted or unsubstituted linear, branched, or cyclic C₁-C₆ alkyl, or substituted or unsubstituted linear, branched, or cyclic C₁-C₆ alkoxy; each R⁵, R⁶, R⁷, and R⁸ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl.

The choice of the anion in the ionic liquid can be particularly relevant to the rate and level of biopolymer dissolution. While not wishing to be bound by theory, the primary mechanism of solvation of carbohydrates by an ionic liquid is the anion's ability to break the extensive hydrogen-bonding networks by specific interactions with hydroxyl groups. Thus, it is believed that that the dissolution of biopolymer (e.g., chitin, cellulose) is enhanced by increasing the hydrogen bond acceptance and basicity of the anion. For example, by using anions that can accept hydrogen bonds and that are relatively basic, one can not only dissolve pure biopolymer, but one can dissolve practical grade biopolymers and even extract a biopolymer from raw biomass, as described herein. Accordingly, in some examples, the anions are substituted or unsubstituted acyl units R¹⁰CO₂, for example, formate HCO₂⁻, acetate CH₃CO₂⁻ (also noted herein as [OAc]), proprionate, CH₃CH₂CO₂⁻, butyrate CH₃CH₂CH₂CO₂⁻, and benzylate, C₆H₅CO₂⁻; substituted or unsubstituted sulfates: (R¹⁰O)S(═O)₂O⁻; substituted or unsubstituted sulfonates R¹⁰SO₃⁻, for example (CF₃)SO₃; substituted or unsubstituted phosphates: (R¹⁰O)₂P(═O)O⁻; and substituted or unsubstituted carboxylates: (R¹⁰O)C(═O)O⁻. Non-limiting examples of R¹⁰ include hydrogen; substituted or unsubstituted linear branched, and cyclic alkyl; substituted or unsubstituted linear, branched, and cyclic alkoxy; substituted or unsubstituted aryl; substituted or unsubstituted aryloxy; substituted or unsubstituted heterocyclic; substituted or unsubstituted heteroaryl; acyl; silyl; boryl; phosphino; amino; thio; and seleno. In some examples, the anion is C₁₋₆ carboxylate.

Still further examples of anions are deprotonated amino acids, for example, Isoleucine, Alanine, Leucine, Asparagine, Lysine, Aspartic Acid, Methionine, Cysteine, Phenylalanine, Glutamic Acid, Threonine, Glutamine, Tryptophan, Glycine, Valine, Proline, Selenocysteine, Serine, Tyrosine, Arginine, Histidine, Ornithine, Taurine.

It is also contemplated that other anions can be used in some instances, such as halides, (i.e., F⁻, Cl⁻, Br⁻, and I⁻), CO₃²⁻; NO₂⁻, NO₃⁻, SO₄²⁻, CN⁻, arsenate(V), AsX₆; AsF₆, and the like; stibate(V) (antimony), SbX₆; SbF₆, and the like.

Other non-limiting examples of ionic liquid anions include substituted azolates, that is, five membered heterocyclic aromatic rings that have nitrogen atoms in either positions 1 and 3 (imidazolates); 1, 2, and 3 (1,2,3-triazolates); or 1, 2, 4 (1, 2, 4-triazolate). Substitutions to the ring occur at positions that are not located in nitrogen positions (these are carbon positions) and include CN (cyano-), NO₂ (nitro-), and NH₂ (amino) group appended to the heterocyclic azolate core.

In some examples of suitable ionic liquids, an anion is chosen from formate, acetate, propionate, butyrate, (CF₃)SO₃⁻, (R¹⁰O)S(═O)₂O⁻; (R¹⁰O)₂P(═O)O⁻; (R¹⁰O)C(═O)O⁻; and R¹⁰CO₂⁻; each R¹⁰ is independently C₁-C₆ alkyl. The anion portion of the ionic liquid can be written without the charge, for example, OAc, CHO₂, Cl, Br, RCH₃OPO₂, and PF₆.

In some examples, wherein the ionic liquid comprises a cation and an anion, wherein the cation is selected from the group consisting of:

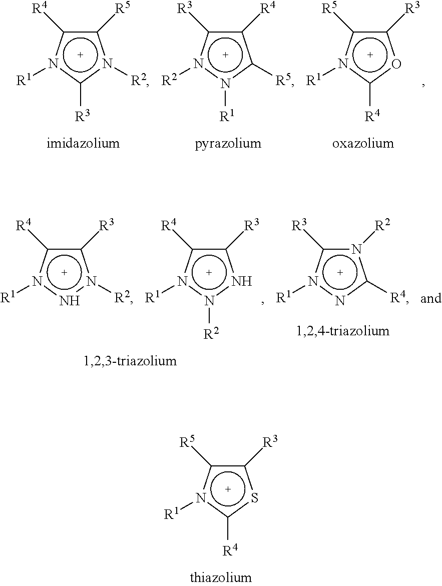

where each R¹ and R² is, independently, a substituted or unsubstituted linear, branched, or cyclic C₁-C₆ alkyl, or substituted or unsubstituted linear, branched, or cyclic C₁-C₆ alkoxy; each R³, R⁴, and R⁵ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl; and
wherein the anion is selected from the group consisting of C₁₋₆ carboxylate, halide, CO₃²; NO₂⁻, NO₃⁻, SO₄²⁻, CN⁻, R¹⁰CO₂, (R¹⁰O)₂P(═O)O, (R¹⁰O)S(═O)₂O, or (R¹⁰O)C(═O)O; where R¹⁰ is hydrogen; substituted or unsubstituted linear, branched, or cyclic alkyl; substituted or unsubstituted linear, branched, or cyclic alkoxy; substituted or unsubstituted aryl; substituted or unsubstituted aryloxy; substituted or unsubstituted heterocyclic; and substituted or unsubstituted heteroaryl.

In some examples, the ionic liquid contains an aromatic cation. In some examples, the ionic liquid contains an imidazolium cation. In some examples, the ionic liquid is a 1-alkyl-3-alkyl imidazolium C₁-C₆ carboxylate or a 1-alkyl-3-alkyl imidazolium C₁-C₆ carboxylate halide. In some examples, the ionic liquid is 1-ethyl-3-methyl-imidazolium acetate ([C₂mim]OAc), 1-butyl-3-methyl-imidazolium chloride ([C₄mim]Cl).

Any ionic liquid that effectively dissolves the biopolymer and graphene can be used in the methods disclosed herein. What is meant by “effectively dissolves” is 25% by weight or more of the chitin present is solubilized (e.g., 45% or more, 60% or more, 75% or more, or 90% or more). The formulator can select the ionic liquid for use in the disclosed methods by the one or more factors, for example, solubility of the biopolymer and/or graphene.

It is further understood that the disclosed ionic liquids can include solvent molecules (e.g., water); however, these solvent molecules are not required to be present in order to form the ionic liquids. That is, these compositions can contain, at some point during preparation and application no or minimal amounts of solvent molecules that are free and not bound or associated with the ions present in the ionic liquid composition.

The disclosed ionic liquids can be substantially free of water in some examples (e.g., immediately after preparation of the compositions and before any further application of the compositions). By substantially free is meant that water is present at less than 10, 9, 8, 7, 6, 5, 4, 3, 2, 1, 0.5, 0.25, or 0.1 wt. %, based on the total weight of the composition.

The ionic liquids can, after preparation, be further diluted with solvent molecules (e.g., water) to form a solution suitable for application. Thus, the disclosed ionic liquids can be liquid hydrates, solvates, or solutions. It is understood that solutions formed by diluting ionic liquids, for example, possess enhanced chemical properties that are unique to ionic liquid-derived solutions.

By the term “biopolymer” is meant herein any one or more of cellulose, hemicelluloses, chitin, chitosan, silk, or lignin. For example, the biopolymer can comprise a cellulose-rich material which comprises primarily cellulose, but also has some lignin and hemicellulose content.

Cellulose is the most abundant polymer on Earth and enormous effort has been put into understanding its structure, biosynthesis, function, and degradation (Stick, R. V. Carbohydrates—The Sweet Molecules of Life, 2001, Academic Press, New York.). Cellulose is actually a polysaccharide consisting of linear chain of several hundred to over ten thousand β(1→4) linked D-glucose units. The chains are hydrogen bonded either in parallel or anti-parallel manner which imparts more rigidity to the structure, and a subsequent packaging of bound-chains into microfibrils forms the ultimate building material of the nature.

Hemicellulose is the principal non-cellulosic polysaccharide in lignocellulosic biomass. Hemicellulose is a branched heteropolymer comprising different sugar monomers with 500-3000 units. Hemicellulose is usually amorphous and has higher reactivity than the glucose residue because of different ring structures and ring configurations. Lignin is the most complex naturally occurring high-molecular weight polymer. Lignin relatively hydrophobic and aromatic in nature, but lacks a defined primary structure.

Chitin is an N-acetyl-D-glucosamine polymer that has a similar structure to cellulose. It is the most abundant polymer in the marine environment. Chitin is the main component of the exoskeletons of arthropods, such as crustaceans and in the cell walls of fungi. It has been a major source of surface pollution in coastal areas. Both chitin and its major derivative chitosan (obtained by deacetylation of chitin) have numerous applications. The bioactivity, biocompatibility, and low toxicity of native or chemically-modified chitin and chitosan make them suitable for controlled drug release, cosmetics, food preservation, fertilizer, or biodegradable packaging materials, or waste water processing and other industrial applications. Chitin, however, is highly hydrophobic and is insoluble in water and most organic solvents due to the high density of hydrogen bonds of the adjacent chains in solid state. The difficulty in the dissolution restricts the use of chitin as a replacement for synthetic polymers.

Crustacean shells are currently the major source of chitin available for industrial processing. The best characterized sources of chitin are shellfish (including shrimp, crab, lobster, and krill), oyster, and squids. Annual synthesis of chitin in freshwater and marine ecosystem is about 600 and 1600 million tons, respectively. Producing chitin in industry is primarily from the exoskeletons of marine crustacean shell waste by a chemical method that involves acid demineralization, alkali deproteinization, followed by decolorization. Even though the current industrialized chemical process isolates chitin from crustacean shells efficiently, disadvantages exist in these procedures, including the use of corrosive acids, bases, and strong oxidants which are not environmentally friendly. In addition, these processes can modify or nullify the desired physiochemical properties of chitin, for example, by acid demineralization, shorting the chitin chain length, as well as, degrading the chitin during deproteinization in hot alkali solutions. These undesired changes in the properties of chitin can have a profound affect when the chitin obtained therefrom must have specific molecular weight distributions and degrees of acetylation (DA).

In some examples, contacting the ionic liquid with the biopolymer comprises dissolving or dispersing at least a portion of a source of the biopolymer in the ionic liquid. In some examples, the source of the biopolymer can comprise a biomass. For example, the disclosed methods can be used to extract a wide variety of biopolymers from various biomasses. The disclosed methods can make use of various types of biomass and thereby solubilize various biopolymers therefrom. The term “biomass,” as used herein, refers to living or dead biological material that can be used in one or more of the disclosed methods. In the disclosed methods the “biomass” can comprise any cellulosic, lignocellulosic, and/or chitinous biomass and can include materials comprising cellulose, chitin, chitosan, and optionally hemicellulose, lignin, starch, oligosaccharides and/or monosaccharides, their mixtures, and breakdown products (e.g., metabolites). Biomass can also comprise additional components, such as protein and/or lipid. Biomass can be derived from a single source, or biomass can comprise a mixture derived from more than one source. Some specific examples of suitable biomasses that can be used in the disclosed methods include, but are not limited to, bioenergy crops, agricultural residues, municipal solid waste, industrial solid waste, sludge from paper manufacture, yard waste, wood, and forestry waste. Additional examples of suitable types of biomass include, but are not limited to, corn grain, corn cobs, crop residues such as corn husks, corn stover, grasses, wheat, wheat straw, hay, rice straw, switchgrass, waste paper, sugar cane bagasse, sorghum, soy, components obtained from milling of grains, trees (e.g., pine), branches, roots, leaves, wood chips, wood pulp, sawdust, shrubs and bushes, vegetables, fruits, flowers, animal manure, multi-component feed, and crustacean biomass (i.e., chitinous biomass).

Lignocellulosic biomass typically comprises of three major components: cellulose, hemicellulose, and lignin, along with some extractive materials (Sjostorm, E. Wood Chemistry: Fundamentals and Applications, 2nd ed., 1993, New York.). Depending on the source, their relative compositions usually vary to certain extent. The lignocellulosic biomass can, in some examples, be chosen from softwood or hardwood. Softwood lignin primarily comprises guaiacyl units, and hardwood lignin comprises both guaiacyl and syringyl units. Cellulose content in both hardwood and softwood is 43±2%. Typical hemicellulose content in wood is 28-35 wt %, depending on type of wood. Lignin content in hardwood is 18-25% while softwood may contain 25-35% of lignin. While each of these components could be used in a wide variety of applications including synthesis of platform and commodity chemicals, materials, and production of energy, these components can rarely be separated from biomass in their original form. The principal reason has been the need of a universal processing media for biomass. The components of lignocellulosic biomass are held together by primary lignocellulosic bonds. Lignocellulosic bonds are varied in nature and typically comprise cross-linked networks. Traditionally, lignocellulosic biomass cannot be dissolved without degrading in any conventional solvents, and it can be difficult to separate these components in a pure form. However, immense possibilities of separated lignin and hemicellulose-based products have been widely studied. The impact of different process options to convert renewable lignocellulosic feedstocks into valuable chemicals and polymers has been summarized by Gallezot (Green Chem. 2007, 9, 295-302, which is incorporated by reference herein in its entirety for its teaching of feedstock processing.).

Chitinous biomass can, in some examples, comprise an arthropod biomass, a fungi biomass, or a combination thereof. An arthropod biomass can, for example, comprise the exoskeleton of an arthropod chosen from shrimp, prawn, crayfish, crab, lobster, insect, and combinations thereof. In some examples, the chitinous biomass can contain chitin and non-chitin material.

In some examples, the source of chitin is pure chitin, for example, pure chitin obtained from crab shells, C9752, available from Sigma, St. Louis, Mo. In other examples, the source of chitin is practical grade chitin obtained from crab shells, C7170, available from Sigma, St. Louis, Mo. In further examples, the source of chitin is chitinous biomass, such as shrimp shells that are removed from the meat by peeling and processed to insure all shrimp meat is removed. However, any biomass comprising chitin or mixtures of chitin and chitosan, or mixtures of chitin, chitosan, and other polysaccharides can be used as the source of chitin.

When contemplating the biomass or source of chitin, the formulator can take into consideration the amount of chitin that comprises the biomass or source of chitin. For example, “pure chitin” can comprise from 75% to 85% by weight of chitin. “Technical grade” or “practical grade” chitin can comprise from 70% to 80% by weight of chitin. As it relates to crude biomass sources, one example of shrimps skins or shells comprises 27.2% chitin by weight, while, one example of crab shells comprises 23.9% chitin by weight.

Chitin derived from crustaceans is available from suppliers as “pure chitin” and as “practical grade chitin” and can be used herein. These forms of chitin undergo a process similar to the Kraft Process for obtaining cellulose from wood or other sources of cellulose. During the process of preparing pure chitin and practical grade chitin, there is a breakdown of the polysaccharide chains such that the resulting chitin has a shorter chain length and therefore a lower average molecular weight than it had before it was processed. Consequently, the separated chitin obtained when using the disclosed methods with these sources of chitin will likewise be of lower molecular weight than had the disclosed methods been followed with unprocessed chitinous biomass. Nonetheless, it can still be useful in various circumstances to use pure or practical grade chitin in the disclosed methods. Thus, in certain examples of the disclosed methods, the source of chitin can be pure or practical grade chitin.

One benefit of the disclosed methods, however, is that chitin can be obtained directly from chitinous biomass. As such, the disclosed methods provide a method of directly extracting chitin from a chitinous biomass without substantially shortening the polysaccharide chains. As such, the disclosed methods provides a unique method for obtaining polymeric materials comprising chitin that has the original full polysaccharide chain length (and molecular weight). Moreover the chitin can be substantially free of agents that are typically found in pure and practical grade chitin, such as methanesulfonic acid, trichloroacetic acid, dichloroacetic acid, formic acid, and dimethylacetamide. Thus, in certain examples of the disclosed methods, the source of chitin can be chitinous biomass.

The concentration of biopolymer in the mixture can, for example, be 0.1 wt % or more with respect to the weight of the ionic liquid (e.g., 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 6 wt % or more, 7 wt % or more, 8 wt % or more, 9 wt % or more, 10 wt % or more, 15 wt % or more, 20 wt % or more, or 25 wt % or more). In some examples, the concentration of the biopolymer in the mixture can be 30 wt % or less with respect to the weight of the ionic liquid (e.g., 25 wt % or less, 20 wt % or less, 15 wt % or less, 10 wt % or less, 9 wt % or less, 8 wt % or less, 7 wt % or less, 6 wt % or less, 5 wt % or less, 4.5 wt % or less, 4 wt % or less, 3.5 wt % or less, 3 wt % or less, 2.5 wt % or less, 2 wt % or less, 1.5 wt % or less, 1 wt % or less, or 0.5 wt % or less). The concentration of the biopolymer in the mixture can range from any of the minimum values described above to any of the maximum values described above. For example, the concentration of the biopolymer in the mixture can be from 0.1 to 30 wt % with respect to The weight of the ionic liquid (e.g., from 0.1 wt % to 15 wt %, from 15 wt % to 30 wt %, from 0.1 wt % to 10 wt %, from 10 wt % to 20 wt %, from 20 wt % to 30 wt %, or from 0.1 wt % to 20 wt %).

In some examples, the biopolymer source can be dissolved or dispersed in the ionic liquid at a temperature of 25° C. or more (e.g., 30° C. or more, 35° C. or more, 40° C. or more, 45° C. or more, 50° C. or more, 60° C. or more, 70° C. or more, 80° C. or more, 90° C. or more, 100° C. or more, 110° C. or more, 120° C. or more, 130° C. or more, 140° C. or more, 150° C. or more, 160° C. or more, 170° C. or more, or 180° C. or more). In some examples, the biopolymer source can be dissolved or dispersed in the ionic liquid at a temperature of 190° C. or less (e.g., 180° C. or less, 170° C. or less, 160° C. or less, 150° C. or less, 140° C. or less, 130° C. or less, 120° C. or less, 110° C. or less, 100° C. or less, 90° C. or less, 80° C. or less, 70° C. or less, 60° C. or less, 50° C. or less, 45° C. or less, 40° C. or less, 35° C. or less, or 30° C. or less). The temperature at which the biopolymer source is dissolved or dispersed in the ionic liquid can range from any of the minimum values described above to any of the maximum values described above. For example, the biopolymer source can be dissolved or dispersed in the ionic liquid at a temperature of from 25° C. to 190° C. (e.g., from 25° C. to 100° C., from 100° C. to 190° C., from 25° C. to 60° C., from 60° C. to 100° C., from 100° C. to 140° C., from 140° C. to 190° C., from 30° C. to 180° C., or from 25° C. to 40° C.).

The term “graphene,” as used herein, refers to planar materials that include from one to several atomic monolayers of sp²-bonded carbon atoms. Graphene can have a thickness of from 1 to 100 carbon layers (e.g., from 1 to 80 graphene layers, from 1 to 60 graphene layers, from 1 to 40 graphene layers, or from 1 to 20 graphene layers). The graphene can have an average thickness, for example, of from 0.3 nm to 55 nm (e.g., from 0.3 nm to 50 nm, from 0.3 nm to 45 nm, from 0.3 nm to 40 nm, from 0.3 nm to 35 nm, from 0.3 nm to 30 nm, from 0.3 nm to 25 nm, from 0.3 nm to 20 nm, from 0.3 nm to 15 nm, from 0.3 nm to 10 nm, or from 0.3 nm to 5 nm). The term “graphene,” as used herein can thus include a wide range of graphene-based materials including, for example, graphene oxide, graphite oxide, chemically converted graphene, functionalized graphene, functionalized graphene oxide, functionalized graphite oxide, functionalized chemically converted graphene, and combinations thereof. The purity of the graphene can be determined using various techniques, i.e. by phase contrast transmission electron microscopy, X-ray diffraction analysis, Raman spectroscopy, thermal gravimetric analysis, or any combination thereof. In some embodiments, graphene is substantially planar and thus not a nanotube, nanorod, or sphere.

The concentration of graphene in the mixture can, for example, be 0.01 wt % or more compared to the amount of biopolymer in the mixture (e.g., 0.05 wt % or more, 0.1 wt % or more, 0.5 wt % or more, 1 wt % or more, 2 wt % or more, 3 wt % or more, 4 wt % or more, 5 wt % or more, 6 wt % or more, 7 wt % or more, 8 wt % or more, or 9 wt % or more). In some examples, the concentration of graphene in the mixture can be 10 wt % or less compared to the amount of biopolymer in the mixture (e.g., 9 wt % or less, 8 wt % or less, 7 wt % or less, 6 wt % or less, 5 wt % or less, 4 wt % or less, 3 wt % or less, 2 wt % or less, 1 wt % or less, 0.5 wt % or less, 0.1 wt % or less, or 0.05 wt % or less). The concentration of graphene in the mixture can range from any of the minimum values described above to any of the maximum values described above. For example, the concentration of the graphene in the mixture can be from 0.01 to 10 wt % compared to the amount of biopolymer in the mixture (e.g., from 0.01 wt % to 5 wt %, from 0.5 wt % to 10 wt %, from 0.01 wt % to 2 wt %, from 2 wt % to 4 wt %, from 4 wt % to 6 wt %, from 6 wt % to 8 wt %, from 8 wt % to 10 wt %, or from 0.01 wt % to 4 wt %).

In some examples, the graphene is the minor component such that the biopolymer supports the graphene. In other examples, the biopolymer is the minor component such that the graphene supports the biopolymer. For example, the concentration of graphene in the mixture can be 10 wt % or more compared to the amount of biopolymer in the mixture (e.g., 15 wt % or more, 20 wt % or more, 25 wt % or more, 30 wt % or more, 35 wt % or more, 40 wt % or more, 45 wt % or more, 50 wt % or more, 55 wt % or more, 60 wt % or more, 65 wt % or more, 70 wt % or more, 75 wt % or more, 80 wt % or more, 85 wt % or more, or 90 wt % or more). In some examples, the concentration of the graphene in the mixture can be less than 100 wt % compared to the amount of biopolymer in the mixture (e.g., 95 wt % or less, 90 wt % or less, 85 wt % or less, 80 wt % or less, 75 wt % or less, 70 wt % or less, 65 wt % or less, 60 wt % or less, 55 wt % or less, 50 wt % or less, 45 wt % or less, 40 wt % or less, 35 wt % or less, 30 wt % or less, 25 wt % or less, 20 wt % or less, or 15 wt % or less). The concentration of graphene in the mixture can, in certain examples, range from any of the minimum values described above to any of the maximum values described above. For example, the concentration of the graphene in the mixture can be from 10 wt % to less than 100 wt % compared to the amount of biopolymer in the mixture (e.g., from 10 wt % to 50 wt %, from 50 wt % to less than 100 wt %, from 10 wt % to 30 wt %, from 30 wt % to 50 wt %, from 50 wt % to 70 wt %, from 70 wt % to 90 wt %, from 90 wt % to less than 100 wt %, or from 60 wt % to 90 wt %).

Any suitable form of graphene or graphitic material (e.g., graphene architecture) can be used. Suitable forms of graphene are known in the art, and can be obtained commercially or prepared according to known methods. For example, the graphene can comprise graphene flakes, graphene sheets, graphene ribbons, or graphene particles; the graphene can comprise a graphene architecture (e.g., material comprising graphene) such as graphene nanotubes; or combinations thereof. In some examples, the graphene can comprise graphene flakes which have a thickness and an average maximum lateral dimension. In some examples, the average maximum lateral dimension of the graphene flakes can be 1 nm or more (e.g., 5 nm or more, 10 nm or more, 15 nm or more, 20 nm or more, 30 nm or more, 40 nm or more, 50 nm or more, 75 nm or more, 100 nm or more, 125 nm or more, 150 nm or more, 200 nm or more, 250 nm or more, 300 nm or more, 350 nm or more, 400 nm or more, 500 nm or more, 600 nm or more, 700 nm or more, 800 nm or more, 900 nm or more, 1 μm or more, 2 μm or more, 3 μm or more, 4 μm or more, 5 μm or more, 10 μm or more, 15 μm or more, 20 μm or more, 30 μm or more, 40 μm or more, 50 μm or more, 60 μm or more, 70 μm or more, 80 μm or more, or 90 μm or more). In some examples, the average maximum lateral dimension of the graphene flaked can be 100 μm or less (e.g., 90 μm or less, 80 μm or less, 70 μm or less, 60 μm or less, 50 μm or less, 40 μm or less, 30 μm or less, 20 μm or less, 15 μm or less, 10 μm or less, 5 μm or less, 4 μm or less, 3 μm or less, 2 μm or less, 1 μm or less, 900 nm or less, 800 nm or less, 700 nm or less, 600 nm or less, 500 nm or less, 400 nm or less, 350 nm or less, 300 nm or less, 250 nm or less, 200 nm or less, 150 nm or less, 125 nm or less, 100 nm or less, 75 nm or less, 50 nm or less, 40 nm or less, 30 nm or less, 20 nm or less, 15 nm or less, 10 nm or less, or 5 nm or less). The average maximum lateral dimension of the graphene flakes can range from any of the minimum values described above to any of the maximum values described above. For example, the average maximum lateral dimension of the graphene flakes can be from 1 nm to 100 μm (e.g., from 1 nm to 50 μm, from 50 μm to 100 μm, from 1 nm to 50 nm, from 50 nm to 100 nm, from 100 nm to 500 nm, from 500 nm to 1 μm, from 1 μm to 50 μm, or from 1 nm to 25 μm).

In some examples, contacting the ionic liquid with the biopolymer and graphene comprises contacting the ionic liquid with the graphene to form a precursor mixture and contacting the precursor mixture with the biopolymer to form the mixture. In some examples, the ionic liquid is contacted with the graphene under agitation and/or the precursor mixture is contacted with the biopolymer under agitation. The agitation can, for example, comprise sonicating, stirring, or a combination thereof. The methods can, for example, further comprise heating the precursor mixture at a temperature of 25° C. or more (e.g., 30° C. or more, 35° C. or more, 40° C. or more, 45° C. or more, 50° C. or more, 60° C. or more, 70° C. or more, 80° C. or more, 90° C. or more, 100° C. or more, 110° C. or more, 120° C. or more, 130° C. or more, 140° C. or more, 150° C. or more, 160° C. or more, 170° C. or more, or 180° C. or more). In some examples, the precursor mixture can be heated at a temperature of 190° C. or less (e.g., 180° C. or less, 170° C. or less, 160° C. or less, 150° C. or less, 140° C. or less, 130° C. or less, 120° C. or less, 110° C. or less, 100° C. or less, 90° C. or less, 80° C. or less, 70° C. or less, 60° C. or less, 50° C. or less, 45° C. or less, 40° C. or less, 35° C. or less, or 30° C. or less). The temperature at which the precursor mixture is heated can range from any of the minimum values described above to any of the maximum values described above. For example, the precursor mixture can be heated at a temperature of from 25° C. to 190° C. (e.g., from 25° C. to 100° C., from 100° C. to 190° C., from 25° C. to 60° C., from 60° C. to 100° C., from 100° C. to 140° C., from 140° C. to 190° C., from 30° C. to 180° C., or from 25° C. to 40° C.).

In some examples, contacting the ionic liquid with the biopolymer and graphene comprises contacting the ionic liquid with the biopolymer to form a precursor mixture and contacting the precursor mixture with the graphene to form the mixture. In some examples, the ionic liquid is contacted with the biopolymer under agitation and/or wherein the precursor mixture is contacted with the graphene under agitation. The agitation can, for example, comprise sonicating, stirring, or a combination thereof. The methods can, for example, further comprise heating the precursor mixture at a temperature of 25° C. or more (e.g., 30° C. or more, 35° C. or more, 40° C. or more, 45° C. or more, 50° C. or more, 60° C. or more, 70° C. or more, 80° C. or more, 90° C. or more, 100° C. or more, 110° C. or more, 120° C. or more, 130° C. or more, 140° C. or more, 150° C. or more, 160° C. or more, 170° C. or more, or 180° C. or more). In some examples, the precursor mixture can be heated at a temperature of 190° C. or less (e.g., 180° C. or less, 170° C. or less, 160° C. or less, 150° C. or less, 140° C. or less, 130° C. or less, 120° C. or less, 110° C. or less, 100° C. or less, 90° C. or less, 80° C. or less, 70° C. or less, 60° C. or less, 50° C. or less, 45° C. or less, 40° C. or less, 35° C. or less, or 30° C. or less). The temperature at which the precursor mixture is heated can range from any of the minimum values described above to any of the maximum values described above. For example, the precursor mixture can be heated at a temperature of from 25° C. to 190° C. (e.g., from 25° C. to 100° C., from 100° C. to 190° C., from 25° C. to 60° C., from 60° C. to 100° C., from 100° C. to 140° C., from 140° C. to 190° C., from 30° C. to 180° C., or from 25° C. to 40° C.).

In some examples, contacting the ionic liquid with the biopolymer and graphene comprises contacting the ionic liquid with the graphene to form a first precursor mixture, contacting the ionic liquid with the biopolymer to form a second precursor mixture, and contacting the first precursor mixture with the second precursor mixture to form the mixture. In some examples, the ionic liquid is contacted with the graphene under agitation, the ionic liquid is contacted with the biopolymer under agitation, the first precursor mixture is contacted with the second precursor mixture under agitation, or a combination thereof. The agitation can, for example, comprise sonicating, stirring, or a combination thereof. The methods can, for example, further comprise heating the first precursor mixture and/or the second precursor mixture at a temperature of 25° C. or more (e.g., 30° C. or more, 35° C. or more, 40° C. or more, 45° C. or more, 50° C. or more, 60° C. or more, 70° C. or more, 80° C. or more, 90° C. or more, 100° C. or more, 110° C. or more, 120° C. or more, 130° C. or more, 140° C. or more, 150° C. or more, 160° C. or more, 170° C. or more, or 180° C. or more). In some examples, the first precursor mixture and/or the second precursor can be heated at a temperature of 190° C. or less (e.g., 180° C. or less, 170° C. or less, 160° C. or less, 150° C. or less, 140° C. or less, 130° C. or less, 120° C. or less, 110° C. or less, 100° C. or less, 90° C. or less, 80° C. or less, 70° C. or less, 60° C. or less, 50° C. or less, 45° C. or less, 40° C. or less, 35° C. or less, or 30° C. or less). The temperature at which the first precursor mixture and/or the second precursor is heated can range from any of the minimum values described above to any of the maximum values described above. For example, the first precursor mixture and/or the second precursor mixture can be heated at a temperature of from 25° C. to 190° C. (e.g., from 25° C. to 100° C., from 100° C. to 190° C., from 25° C. to 60° C., from 60° C. to 100° C., from 100° C. to 140° C., from 140° C. to 190° C., from 30° C. to 180° C., or from 25° C. to 40° C.).

In some examples, the ionic liquid is contacted with the graphene and biopolymer under agitation. The agitation can, for example, comprise sonicating, stirring, or a combination thereof.

The methods can, in some examples, further comprise agitating the mixture. Agitating the mixture can, for example, comprise sonicating the mixture or stirring the mixture.

The methods can, in some examples, further comprise heating the mixture at a temperature of 25° C. or more (e.g., 30° C. or more, 35° C. or more, 40° C. or more, 45° C. or more, 50° C. or more, 60° C. or more, 70° C. or more, 80° C. or more, 90° C. or more, 100° C. or more, 110° C. or more, 120° C. or more, 130° C. or more, 140° C. or more, 150° C. or more, 160° C. or more, 170° C. or more, or 180° C. or more). In some examples, the mixture can be heated at a temperature of 190° C. or less (e.g., 180° C. or less, 170° C. or less, 160° C. or less, 150° C. or less, 140° C. or less, 130° C. or less, 120° C. or less, 110° C. or less, 100° C. or less, 90° C. or less, 80° C. or less, 70° C. or less, 60° C. or less, 50° C. or less, 45° C. or less, 40° C. or less, 35° C. or less, or 30° C. or less). The temperature at which the mixture is heated can range from any of the minimum values described above to any of the maximum values described above. For example, the mixture can be heated at a temperature of from 25° C. to 190° C. (e.g., from 25° C. to 100° C., from 100° C. to 190° C., from 25° C. to 60° C., from 60° C. to 100° C., from 100° C. to 140° C., from 140° C. to 190° C., from 30° C. to 180° C., or from 25° C. to 40° C.).

The methods further comprise contacting the mixture with a non-solvent, thereby forming the graphene-biopolymer composite material in the non-solvent and collecting the graphene-biopolymer composite material from the non-solvent. The graphene can, for example, be substantially homogeneously dispersed throughout the graphene-biopolymer composite material. The graphene-biopolymer composite material can be collected in any manner chosen by the formulator, for example, the graphene-biopolymer composite material can be removed by centrifugation, filtration, or by decanting the non-solvent.

The non-solvent can also be referred to as a coagulant. The non-solvent can, for example, be water, a C₁-C₁₂ linear or branched alcohol, ketone (e.g., acetone or methylethylketone), or a mixture thereof. In some examples, the non-solvent is water, a C₁-C₄ alcohol, ketone, or a mixture thereof. Examples of C₁-C₄ alcohols include, but are not limited to methanol, ethanol, propanol, iso-propanol, butanol, sec-butanol, iso-butanol, or tert-butanol. In some examples, the non-solvent is water.

In some examples, contacting the mixture with non-solvent comprises contacting the mixture with a substrate submerged in the non-solvent, thereby coating the substrate with the composite graphene-biopolymer material. Examples of suitable substrates include, but are not limited to, textiles, plastics, glass, biomedical materials, and the like.

In some examples, the graphene-biopolymer composite material is formed into a fiber, a film, a bead, a mat, or a combination thereof. In some examples, the graphene-biopolymer comprise material is formed into a plurality of fibers, and the plurality of fibers have an average diameter of 8 nm or more (e.g., 10 nm or more, 15 nm or more, 20 nm or more, 30 nm or more, 40 nm or more, 50 nm or more, 75 nm or more, 100 nm or more, 125 nm or more, 150 nm or more, 200 nm or more, 250 nm or more, 300 nm or more, 350 nm or more, 400 nm or more, 500 nm or more, 600 nm or more, 700 nm or more, 800 nm or more, 900 nm or more, 1 μm or more, 2 μm or more, 3 μm or more, 4 μm or more, 5 μm or more, 10 μm or more, 15 μm or more, 20 μm or more, 30 μm or more, 40 μm or more, 50 μm or more, 60 μm or more, 70 μm or more, 80 μm or more, or 90 μm or more). In some examples, the plurality of fibers can have an average diameter of 100 μm or less (e.g., 90 μm or less, 80 μm or less, 70 μm or less, 60 μm or less, 50 μm or less, 40 μm or less, 30 μm or less, 20 μm or less, 15 μm or less, 10 μm or less, 5 μm or less, 4 μm or less, 3 μm or less, 2 μm or less, 1 μm or less, 900 nm or less, 800 nm or less, 700 nm or less, 600 nm or less, 500 nm or less, 400 nm or less, 350 nm or less, 300 nm or less, 250 nm or less, 200 nm or less, 150 nm or less, 125 nm or less, 100 nm or less, 75 nm or less, 50 nm or less, 40 nm or less, 30 nm or less, 20 nm or less, 15 nm or less, or 10 nm or less). The average diameter of the plurality of fibers can range from any of the minimum values described above to any of the maximum values described above. For example, the plurality of fibers can have an average diameter of from 8 nm to 100 μm (e.g., from 8 nm to 50 μm, from 50 μm to 100 μm, from 8 nm to 50 nm, from 50 nm to 100 nm, from 100 nm to 500 nm, from 500 nm to 1 μm, from 1 μm to 50 μm, or from 8 nm to 25 μm).

In some examples, the graphene-biopolymer composite material can be formed into a fiber, a film, a bead, a mat, or a combination thereof by electrospinning, wet jet fiber pulling, film casting, bead preparation, or a combination thereof.

Electrospinning can, for example, be performed at a potential of 15 kV or more (e.g., 16 kV or more, 17 kV or more, 18 kV or more, 19 kV or more, 20 kV or more, 21 kV or more, 22 kV or more, 23 kV or more, 24 kV or more, 25 kV or more, 26 kV or more, 27 kV or more, 28 kV or more, 29 kV or more, 30 kV or more, 31 kV or more, 32 kV or more, 33 kV or more, 34 kV or more, 35 kV or more, 36 kV or more, 37 kV or more, 38 kV or more, or 39 kV or more). In some example, electrospinning can be performed at a potential of 40 kV or less (e.g., 39 kV or less, 38 kV or less, 37 kV or less, 36 kV or less, 35 kV or less, 34 kV or less, 33 kV or less, 32 kV or less, 31 kV or less, 30 kV or less, 29 kV or less, 28 kV or less, 27 kV or less, 26 kV or less, 25 kV or less, 24 kV or less, 23 kV or less, 22 kV or less, 21 kV or less, 20 kV or less, 19 kV or less, 18 kV or less, 17 kV or less, or 16 kV or less). The potential the electrospinning is performed at can range from any of the minimum values described above to any of the maximum values described above. For example, the electrospinning can be performed at a potential of from 15 kV to 40 kV (e.g., from 15 kV to 27 kV, from 27 kV to 40 kV, from 15 kV to 20 kV, from 20 kV to 25 kV, from 25 kV to 30 kV, from 30 kV to 35 kV, from 35 kV to 40 kV, or from 15 kV to 30 kV).

In some examples, the electrospinning can be performed at a flow rate of 50 mL/h or more (e.g., 75 mL/h or more, 100 mL/h or more, 125 mL/h or more, 150 mL/h or more, 175 mL/h or more, 200 mL/h or more, 225 mL/h or more, 250 mL/h or more, or 275 mL/h or more). In some examples, the electrospinning can be performed at a flow rate of 300 mL/h or less (e.g., 275 mL/h or less, 250 mL/h or less, 225 mL/h or less, 200 mL/h or less, 175 mL/h or less, 150 mL/h or less, 125 mL/h or less, 100 mL/h or less, or 75 mL/h or less). The flow rate that the electrospinning is performed at can range from any of the minimum values described above to any of the maximum values described above. For example, the electrospinning can be performed at a flow rate of from 50 mL/h to 300 mL/h (e.g., from 50 mL/h to 175 mL/h, from 175 mL/h to 300 mL/h, from 50 mL/h to 100 mL/h, from 100 mL/h to 150 mL/h, from 150 mL/h to 200 mL/h, from 200 mL/h to 250 mL/h, from 250 mL/h to 300 mL/h, or from 75 mL/h to 275 mL/h).

In some examples, the methods can further comprise separating at least a portion of the ionic liquid from the non-solvent, thereby forming a recycled ionic liquid. The recycled ionic liquid can, in some example, be used to contact the biopolymer and graphene.

Also disclosed herein are compositions comprising the graphene-biopolymer composite materials made by any of the methods described herein. Compositions comprising the graphene-biopolymer composite materials described herein can further include, for example, organic solvents, inorganic solvents, nanoparticles, or any other additive of interest.

Also disclosed herein are articles of manufacture comprising the graphene-biopolymer composite materials made by any of the methods described herein. Examples of articles of manufacture include, for example, conductive textiles, smart fabric, fibers, yearn, and the like.

Also disclosed herein are methods of use of the graphene-biopolymer composite materials made by any of the methods described herein. For example, the graphene-biopolymer composite materials can be used as biodegradable materials for biomedical applications such as scaffolds for tissue regeneration. In some examples, the graphene-biopolymer composite materials can be used as adsorbent materials for metal extraction of filtration systems. In some example, the graphene-biopolymer composite materials can be used as a coating on a substrate.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

Thermally pre-processed shrimp shells (hereafter indicated as “processed”) were received from the Gulf Coast Agricultural and Seafood Cooperative in Bayou La Batre, Ala. The shrimp shells were processed with a screw press and dried at special facility by heating them in fluidized bed dryer to 190° C. The final moisture content after the drying was less than 5 wt %. The thermally dried material was crushed to a particles with 0.635 cm diameter or below by hummer mill and was shipped to The University of Alabama. The received shrimp shells were ground using an electric lab mill (Model M20 S3, IKA™, Wilmington, N.C.) and sieved through a set of four (1000 μm, 500 μm, 250 μm, and 125 μm) brass sieves with wire mesh (Ika Labortechnik, Wilmington, N.C.). The particles size of <125 μm was used for shrimp shell extract and regenerated chitin preparations. Prior to extraction, the ground shrimp shells were dried in oven (Precision Econotherm Laboratory Oven, Winchester, Va.) at 80° C. overnight.

“Raw” shrimp shells (hereafter indicated as “raw”): Frozen shrimp were obtained from Dauphin Island, Mobile County, AL. The shrimp were thawed and peeled to remove visible shrimp meat and the backs of the shells were collected. The shrimp shell backs were washed with tap water (5 times), and then oven-dried at 80° C. for 2 days. The oven-dried shells were ground using a Janke & Kunkel mill (Ika Labortechnik, Wilmington, N.C.) for 5 min followed by sieving through four (1000 μm, 500 μm, 250 μm, and 125 μm) brass sieves with wire mesh (Ika Labortechnik, Wilmington, N.C.), to collect shrimp shell particles with the size <125 μm. Two lbs of thawed shrimp provided ˜26 g of shells.

Ionic liquid (IL) 1-ethyl-3-methylimidazolium acetate ([C₂mim][OAc], >95%) was purchased from IoLitec (Tuscaloosa, Al, USA). Deionized (DI) water was used for all experiments. Graphene nanopowder trial kit (includes the following grades: AO-2, AO-4, AO-3 and C1) was purchased from Graphene Supermarket (graphene-supermarket.com) and used as received.

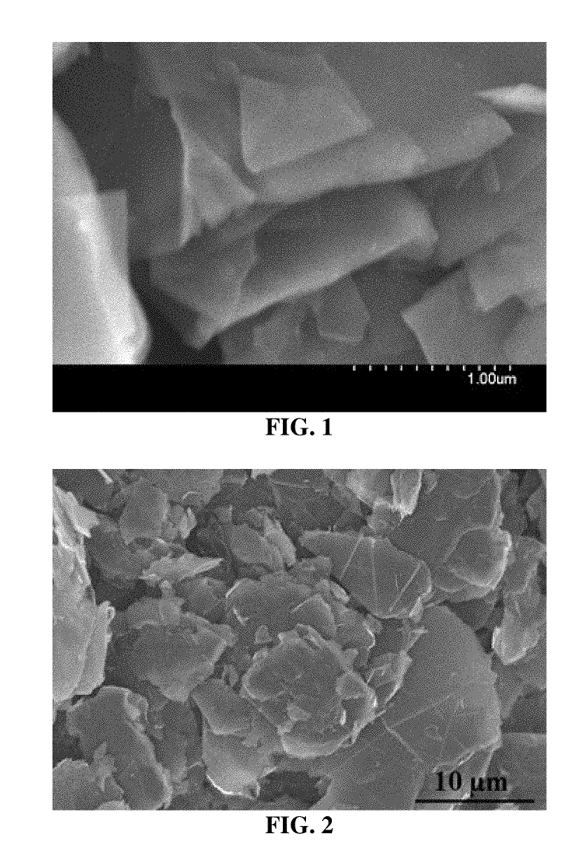

Graphene grade AO-2 was black in color and had a specific surface area of 100 m²/g, a purity of 99.9%, an average flake thickness of 8 nm (20-30 monolayers), and an average particle (lateral) size of ˜550 nm (size distribution ranged from 150-3000 nm). A typical scanning electron microscope (SEM) image of a sample of dry nanopowder of graphene grade AO-2 is shown in FIG. 1.

Graphene grade AO-3 was black in color and had a specific surface area-80 m/g², a purity of 99.2%, an average flake thickness of 12 nm (30-50 monolayers), and an average particle (lateral) size of ˜4500 nm (size distribution ranged from 1500-10000 nm). A typical SEM image of a sample of dry nanopowder of graphene grade AO-3 is shown in FIG. 2.

Graphene grade AO-4 was black in color and had a specific surface area of <15 m²/g, a purity of 98.5%, an average flake thickness of 60 nm, and an average particle (lateral) size of ˜3-7 microns. A typical SEM image of a sample of dry nanopowder of graphene grade AO-4 is shown in FIG. 3.

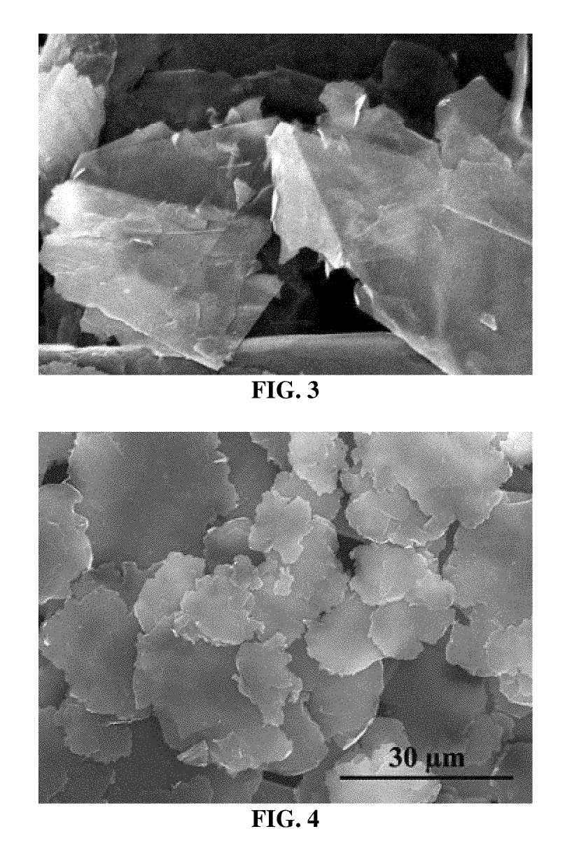

Graphene grade C1 was black in color and had a specific surface area of 60 m/g², a purity of 97%, an average flake thickness of 5-30 nm, and an average particle (lateral) size of ˜5-25 microns. A typical SEM image of a sample of dry nanopowder of graphene grade Cl is shown in FIG. 4.

Powder X-Ray Diffraction (PXRD) was performed using a Bruker D2 Phaser (Bruker Optics Inc.). The angle range (2θ) was from 5 to 40 degrees.

Optical microscopy was performed using a Motic BA 200 Microscope (Carlsbad, Calif.) equipped with an XLI 2.0 camera (XL Imaging, Houston, Tex.) at 40×, 100×. Image analysis was done using XLI-Cap image analysis software.

Scanning Electron Microscopy (SEM) was performed on air-dried mats attached to the Al SEM grids through carbon tape using JEOL-7000 FE-SEM. The surface of the mats was sputter coated with Au prior to imaging. Accelerating voltage was 20 kV with the working distance of 8 nm.

Atomic Force Microscopy (AFM) images were taken in a tapping mode using DI 3100 (Veeco) with the tips (8 nm, 40 N/M) purchased from Mikromasch, USA (Watsonville, Calif.).

### Example 1

Described herein are electrospun biopolymer nanocomposites with graphene-based fillers. Electrospinning is a versatile tool that can design nanofibers with controlled morphology and size for use directly as textile or textile coatings (Bedford N M et al. ACS Appl. Mater. Interfaces 2010, 2, 2448-2455; Varesano A et al. Polym. Int. 2010, 59, 1606-1615). Lately, attention has shifted towards electro spinning biopolymers as bio-friendly alternatives for reasons of environmental sustainability (Shahid-ul-Islam Shahid M and Mohammad F. Ind. Eng. Chem. Res. 2013, 52, 5245-5260). Biopolymers, natural, abundant and underutilized resources (e.g., cellulose, chitin, etc.) are widely available, biodegradable and biocompatible, and thus have several economic and environmental advantages over synthetic polymers. Moreover, the push for environmentally-friendly products creates a demand for technically advantageous materials that can replace petroleum-derived plastics. Among biopolymers, chitin and cellulose are promising because of their high mechanical stability, biocompatibility, and suitability for surface modification (Barber P S et al. Green Chem. 2014, 16, 1828-1836; Qin Y et al. Green Chem. 2010, 12, 968-971).

However, poor solubility of natural biopolymers in commonly used organic solvents makes it challenging to produce networks with nanofibers (Kadokawa J I. RSC Adv. 2015, 5, 12736-12746; Muzzarelli R A A. Mar. Drugs 2011, 9, 1510-1533). Indeed, the strong intermolecular hydrogen bonding of biopolymers can make it challenging to process biopolymers into usable forms. Traditionally, biopolymers have been processed by solution methods in specific solvent systems. For example, to disrupt the strong inter- and intramolecular hydrogen-bonds between chitin polymer chains, harsh chemicals such as lithium chloride/dimethylacetamide (LiCl/DMAc), methanesulphonic acid, hexafluoroisopropanol (HFIP), and sodium hydroxide/urea are used (Li G et al. Carbohydr. Polymer 2010, 80, 970-976; Zhong C et al. Soft Matter 2010, 6, 5298-5301). Among those solvents, hexafluoroisopropanol was used for electrospinning of chitin that resulted in mats with wide (40 to 600 nm) fiber size distribution (Min B M et al. Polymer 2004, 45, 7137-7142). Apart from solvent toxicity, electrospinning from hexafluoroisopropanol required depolymerization of chitin and a decrease of molecular weight by 10-times prior to electrospinning (Nam J et al. J. Appl. Polym. Sci. 2008, 107, 1547-1554). It has been demonstrated that such molecular weight (MW) decrease substantially diminishes the mechanical properties of materials (Qin Y et al. Green Chem. 2010, 12, 968-971), and the tensile strength of chitin fibers prepared from low molecular weight chitin decreases by a factor of about 2. An alternative is to convert biopolymers into their soluble form, e.g., cellulose acetate from cellulose by acetylation, however, such conversion modifies the inherent properties of the biopolymer.

Ionic Liquids (ILs, salts that are liquid below 100° C., and for particular applications they are liquid below room temperature) offer a unique capability to solubilize biopolymers, including chitin and cellulose, that are insoluble in conventional solvents (Swatloski R P et al. J. Am. Chem. Soc. 2002, 124, 4974-4975; Zhang H et al. Macromolecules 2005, 38, 8272-8277), providing at the same time high thermal stability, low vapor pressure, and conductivity, and therefore providing a potential to replace commonly used electrospinning solvents (Welton T. Chem. Rev. 1999, 99, 2071-2083; Meli L et al. Green Chemistry 2010, 12, 1883-1892). Unlike traditional electrospinning, electrospinning from ionic liquids requires a coagulation bath and solidification of electrospun biopolymers by replacing room temperature ionic liquid with anti-solvent such as water or alcohols. Chitin can be electrospun from ionic liquids (ILs) to form materials with controllable fiber diameter (˜22 nm fibers with ˜7 nm diameter size distribution) and had high surface area. Electrospinning of biopolymers from ionic liquids can also be scaled-up (Shamshina J L et al. ChemSusChem, 2017, 10, 106-111).

Chitin can also be wet-jet spun from ionic liquids (ILs) to form fibers with controllable fiber diameter (Shamshina J L et al. J. Mater. Chem. B, 2014, 25, 3924-3936), and cast into films (King C et al. Green Chem, 2016, DOI: 10.1039/C6GC02201D). Herein, methods of making biopolymer-graphene composites in a form of fibers, beads, films and electrospun networks is discussed. Methods of coating textile materials with chitin/graphene by directly electrospinning composite solutions onto the solid support is also discussed.

An advantage of using ionic liquids is their ability to simultaneously dissolve biopolymers and stabilize a variety of nanoparticles, including graphite and graphene. Because graphene is rich in π-electrons, strong cation-π interaction can exists between this carbon nanomaterial and an ionic liquid with an aromatic cation, such as an imidazolium cation. The interaction of the ionic liquids with the graphitic surfaces can be influenced by the charge transfer between the component ions (Ghatee M H et al. J. Phys. Chem. C. 2011, 115, 5626-5636). The aromaticity of the cation in the ionic liquid can result in unique charge transfer interactions and enhanced π-interactions with graphene.

The source of the biopolymer is another variable in obtaining a solution of proper viscosity and surface tension. Two biomass sources were investigated in the examples described herein: a) shrimp shell waste (SS) chitin obtained from by direct dissolution in ionic liquid of unprocessed shrimps (Dauphin Island, Mobile County, AL) and b) regenerated purified chitin (regenerated through the dissolution of shrimp shell in ionic liquid, followed by coagulation of the chitin in water, and purification through several washing steps) (Qin Y et al. Green Chem. 2010, 12, 968-971). Several materials with different architectures were prepared from the biopolymers with graphene or graphene oxide, such as electrospun mats, fibers and films.

Preparation of Biomass Solutions

Solutions of “processed” shrimp shell extract were prepared accordingly to a previously published procedure. Briefly, shrimp shells (2 wt %) in [C₂mim][OAc] were prepared by heating using microwave irradiation with 2 sec pulses with manual stirring for 6 min. For the first 30 sec, the heating was done in 10 sec pulses. After the desired microwave time was reached, the solution was transferred into centrifuge tubes and centrifuged at 3000 rpm for 20 min to remove undissolved residues. Centrifuged solutions were poured into tubes (decanted from a residue remained after centrifugation) and were used for obtaining regenerated chitin.

Solutions of raw shrimp shell (“SS”) was provided by 525 solutions, Inc., Tuscaloosa, Ala. The concentration of chitin in solution of shrimp shells was determined by coagulating a shrimp shell solution in deionized water, proper washing and drying and then accessing the concentration using the following equation:

$$wt \% of chitin in S S solution = \frac{mass of obtained oven dry extracted chitin ( g )}{mass of S S solution ( g )}$$

Other solutions with known chitin concentrations were prepared from stock solution by adding required amount of ionic liquids. To ensure proper solutions mixing, the mixtures were heated to 50° C. and kept under stirring overnight (8 h) before electrospinning

To make the solutions of regenerated chitin, the shrimp shell solution of processed biomass (decanted from the residues) as obtained above (60 g for each coagulation) was coagulated in 1 L of deionized water (DI) during constant stirring and left overnight to remove ionic liquid from coagulated chitin. The chitin obtained was transferred into centrifuge tubes to remove any remaining aqueous phase. The fresh DI water was added followed by sonication and centrifugation at 3000 rpm for 15 min. The steps were repeated 10 times. Regenerated chitin was oven dried at 60° C. Regenerated chitin was dried and sieved to obtained chitin particles size <125 μm using the same procedure described above.

Electrospinning

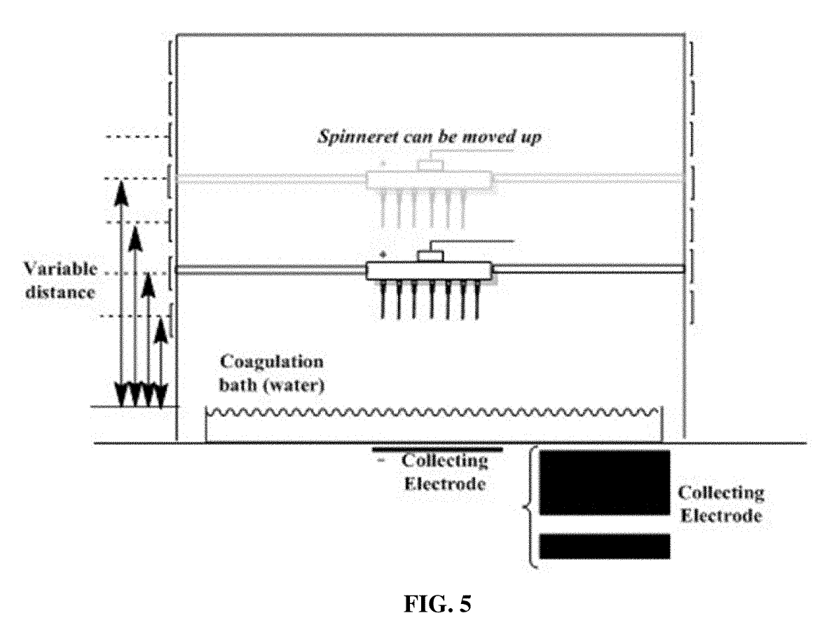

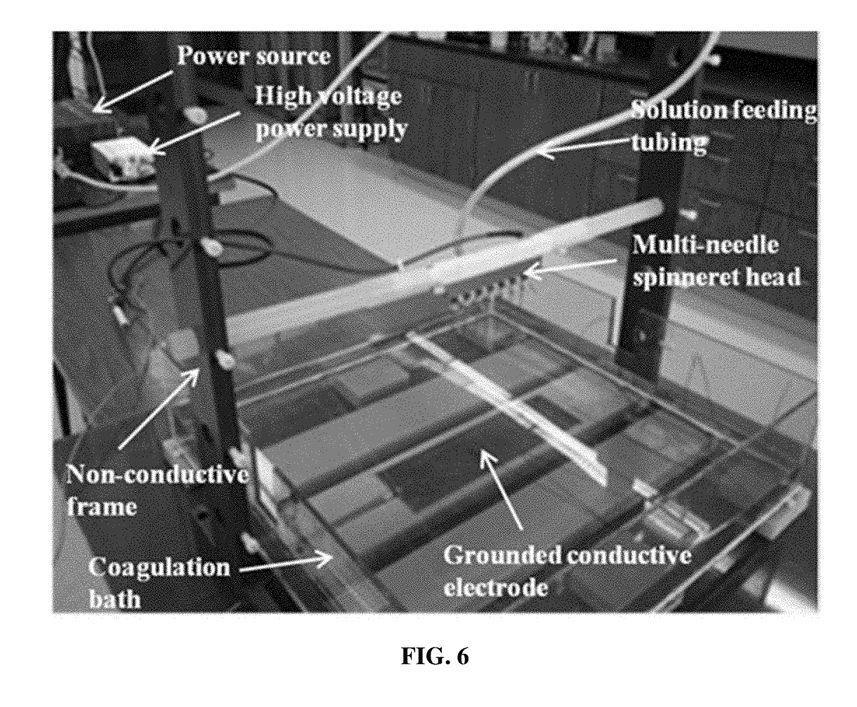

Solutions of shrimp shell and regenerated chitin were electrospun from a custom-built electrospinning system equipped with a multi-needle spinneret as described previously (Shamshina J L et al. ChemSusChem, 2017, 10, 106-111) (FIG. 5 and FIG. 6). Briefly, 50 g chitin solutions in ionic liquid were loaded into a feeding flask directly connected to the spinneret. The spinneret was connected to the high voltage power supply (Ultravolt, USA). An operating voltage was 25-26 kV and solution flow was controlled by gravity in a typical electrospinning experiment. The solutions were electrospun into a coagulation bath filled with deionized (DI) water. The distance between the tips of the needles and coagulation bath was 9 cm. Electrospinning was performed at room temperature. The ionic liquid was removed from the coagulated mats by keeping the mats in pure DI water. The electrospun mats were then air-dried on porous Teflon coated mesh (100 Mesh T304 Stainless 0.0045″ Wire Dia. Green PTFE, Part #100X100S0045W36_PTFE, TVP Inc., Berkeley, Calif., USA)

Graphene/Chitin Composite Solutions

The electrospinning solutions were prepared in two steps. In the first step, the desired concentration of graphene (ranging from 0 to 0.01 wt %) was dispersed in [C₂mim][AOc] ionic liquid by sonication for 12 h. Regenerated chitin was added to the graphene dispersion in ionic liquid and the mixture was heated to 90° C. under constant stirring. The chitin dissolution time was 12-16 h. After chitin dissolution, the composite solution was cooled to room temperature under constant stirring and the room temperature solution was used for electrospinning

A composite solution of shrimp shell chitin and graphene was similarly prepared by first dispersing graphene in ionic liquid, followed by adding the shrimp shell chitin solution into the ionic liquid, to reach the desired chitin concentration. The mixture was heated to 50° C. at constant stirring overnight.

The composite solutions of graphene with shrimp shell (SS) or regenerated chitin were electrospun from a custom-built electrospinning system equipped with a multi-needle spinneret as described previously (Shamshina J L et al. ChemSusChem, 2017, 10, 106-111) (FIG. 5 and FIG. 6). Briefly, the chitin-graphene-ionic liquid composite solution was loaded into a feeding flask directly connected to the spinneret connected to a high voltage power supply (Ultravolt, USA). An operating voltage of 25-26 kV was used. The solution flow was controlled by gravity in a typical electrospinning experiment. The composite solutions were electrospun into a coagulation bath filled with deionized (DI) water. The distance between the tips of the needles and coagulation bath was 9.5 cm. Electrospinning was performed at room temperature. The ionic liquid was removed from the coagulated mats of the composite material by keeping the mats in pure deionized water. The electrospun mats were the air-dried on porous Teflon coated mesh (100 Mesh T304 Stainless 0.0045″ Wire Dia. Green PTFE, Part #100X100S0045W36_PTFE, TVP Inc., Berkeley, Calif., USA)

Electrospinning of Shrimp Shell Solution with Different Graphene Concentrations.

A starting concentration of chitin in shrimp shell solution, 0.4 wt % was used. The graphene concentrations tested were 0.0012 wt %, 0.0054 wt %, and 0.01 wt %, with a grade of graphene marked as AO-4 (thickness 60 nm, lateral size ˜3-7 μm). Electrospinning of the composite shrimp shell solution/graphene solutions at 0.0012 wt %, 0.0054 wt %, and 0.01 wt % graphene concentrations resulted in continuous jet and fiber formation. While electrospinning of composites with 0.0012 wt % and 0.0054 wt % of graphene did not require external pressure (flow by gravity), external pressure was applied for the composites with 0.01 wt % graphene to reach a sufficient flow (Table 1). After the electrospinning, an interconnected fiber network was obtained on the water surface, and the interconnected fiber network was collected and air-dried for further analysis.

TABLE 1
Electrospinning parameters and solution properties of
composite shrimp shell/graphene solution.

| | Graphene | Solution | | | |
|---|---|---|---|---|---|
| Chitin | (AO-4) | con- | | | |
| concentration | concentration | ductivity | Voltage | | |
| (wt %) | (wt %) | (mS/cm) | (kV) | Pressure | Results |
| 0.4 | 0.0012 | — | 24 | gravity | Mat |
| | | | | | formed |
| | 0.0054 | 3.2 | 25 | gravity | Mat |
| | | | | | formed |
| | 0.01 | 3.9 | 25-26 | 1.5 psi | Mat |
| | | | | | formed |

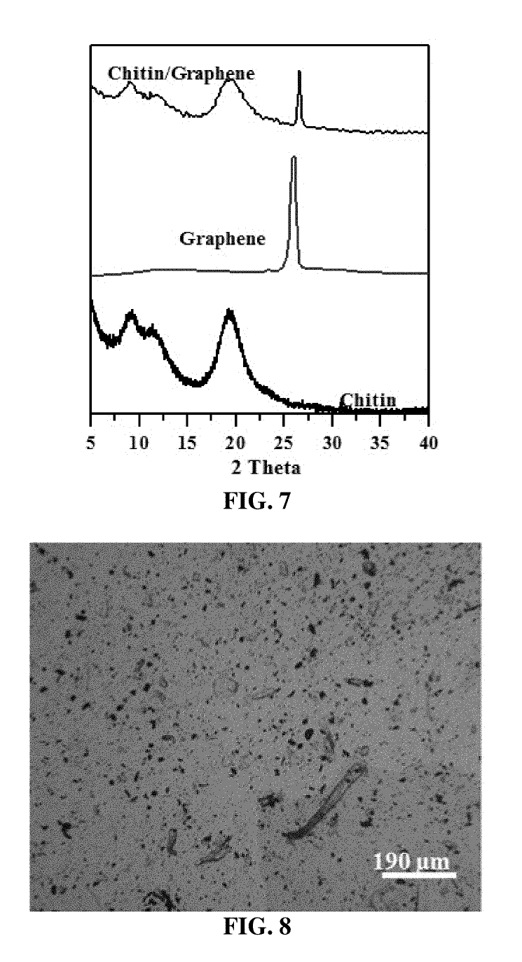

The air-dried mats with graphene concentrations of 0.0054 wt % and 0.01 wt % were grayish as compared to the graphene-free mat and the mat with a graphene concentration of 0.0012 wt %. The grey color of electrospun mats indicates the presence of graphene in the electrospun mats. To confirm the presence of graphene in the mat with 0.0012 wt % graphene concentration, Powder X-Ray Diffraction (PXRD) was taken. As seen from FIG. 7, the composite chitin/graphene mat with 0.0012 wt % of graphene has a peak present at 2θ=27° which corresponds to graphitic carbon. Additionally, the composite chitin/graphene mats have the characteristic peaks of chitin at 2θ=9.3, 12.8 and 19.2°.

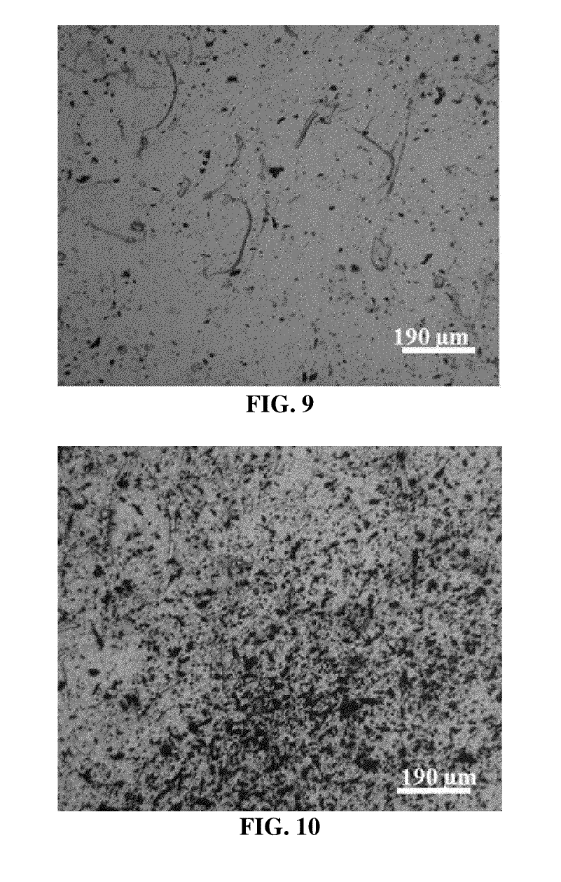

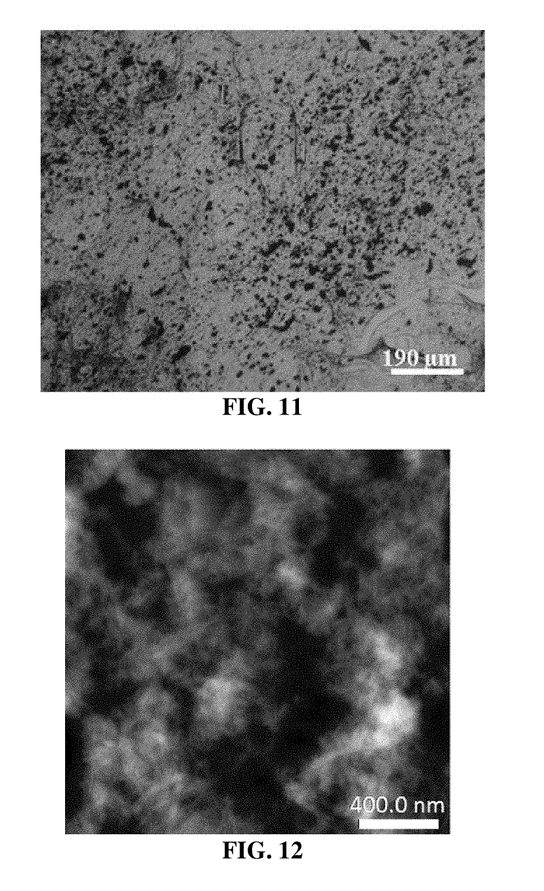

Next, the distribution of graphene flakes within the electrospun composite materials was investigated. For that, the air-dried mats were imaged with an optical microscope. The images obtained for the composites with 0.0012 wt % graphene and 0.01 wt % graphene at different magnifications are presented in FIG. 8-FIG. 11. As can be seen in FIG. 8-FIG. 11, graphene is evenly distributed in the electrospun mats and packing density of the graphene increases as the initial graphene loading in composite solution increases.

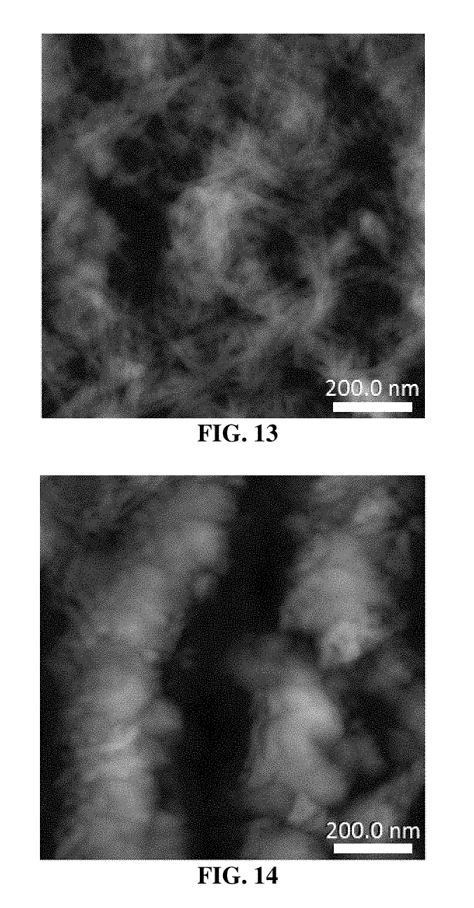

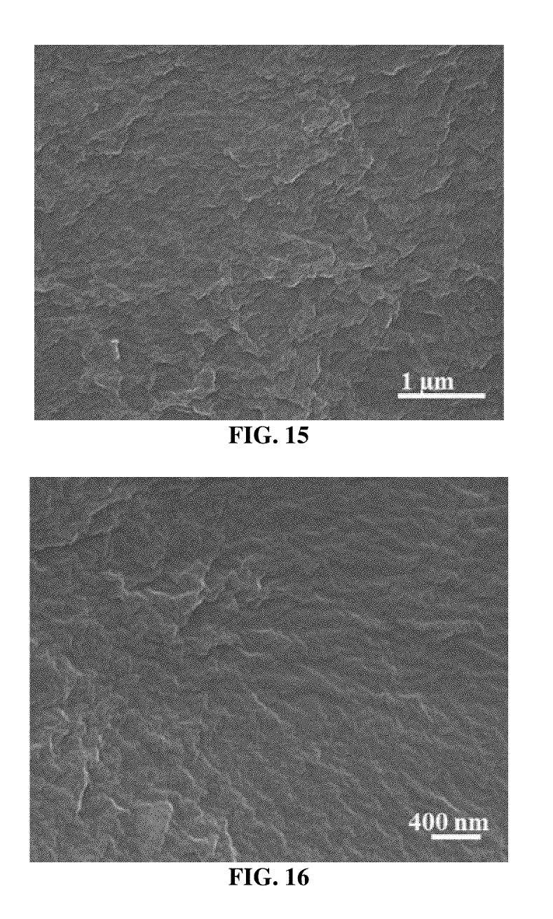

The surface morphology of the electrospun shrimp shell chitin/graphene (AO-4) samples were investigated with atomic force microscopy (AFM) and scanning electron microscopy (SEM). The electrospun shrimp shell chitin/graphene mats with 0.0012 wt % of graphene have a nanofiber morphology with a surface micro-roughness of ˜8 nm, which is ˜1.5 times higher than the roughness of the electrospun shrimp shell chitin mat (i.e., the mat without graphene) (FIG. 12 and FIG. 13). The electrospun composite shrimp shell chitin/graphene mats with 0.0054 wt % of graphene have a rough surface and do not have nanofibers (FIG. 14). The SEM images (FIG. 15 and FIG. 16) of the electrospun shrimp shell chitin/graphene mat with 0.0054 wt % of graphene shows the combination of graphene flakes and nanofibers consistent with AFM imaging.

Electrospinning of Regenerated Chitin with AO-2 and AO-4 as a Source of Graphene.

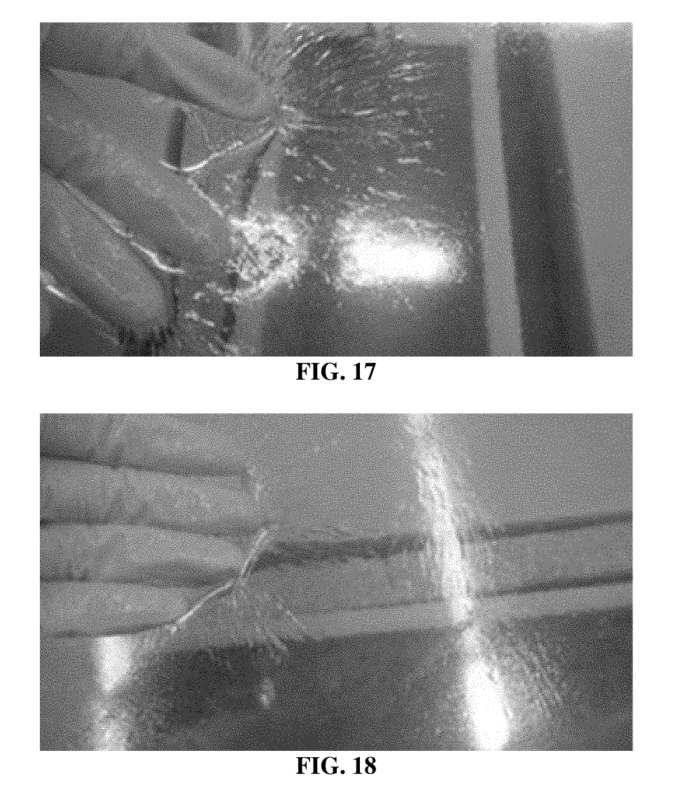

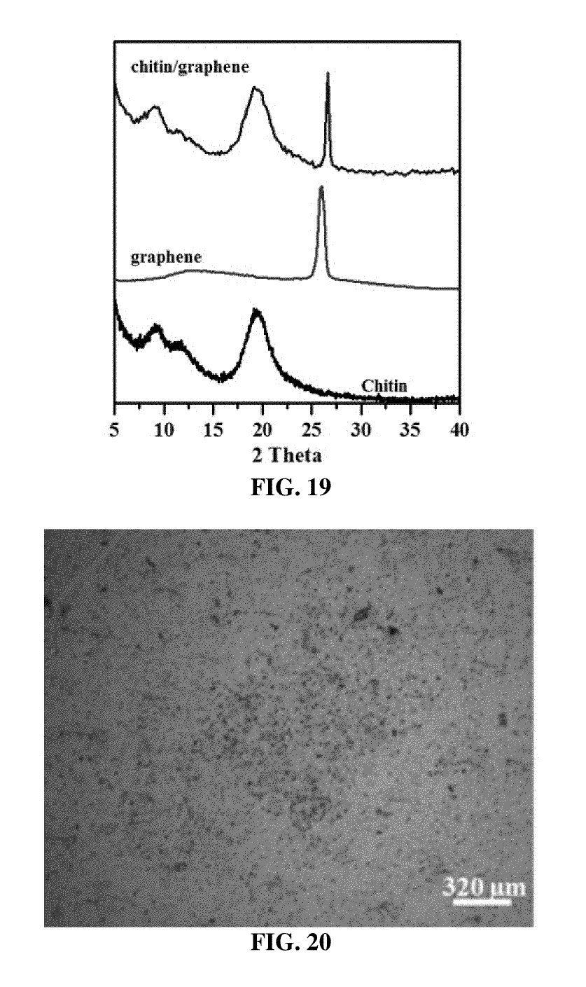

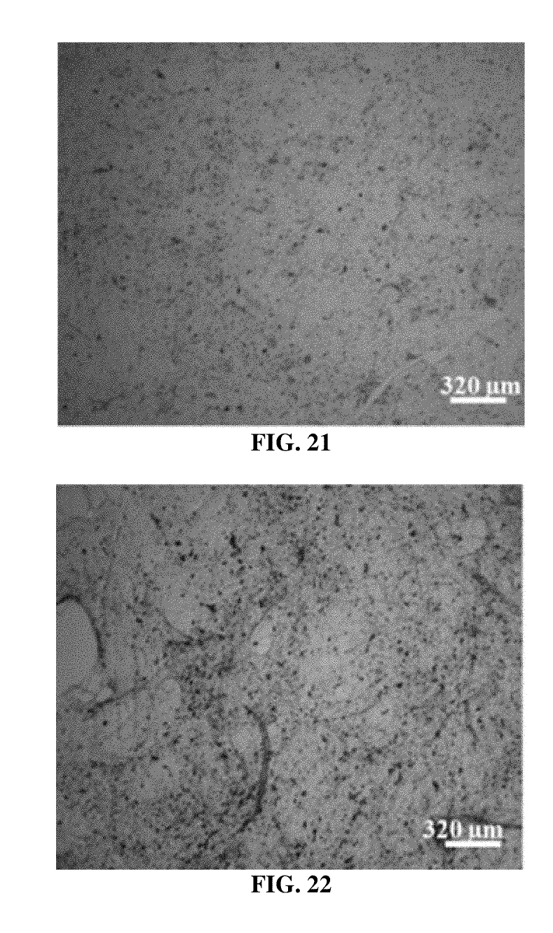

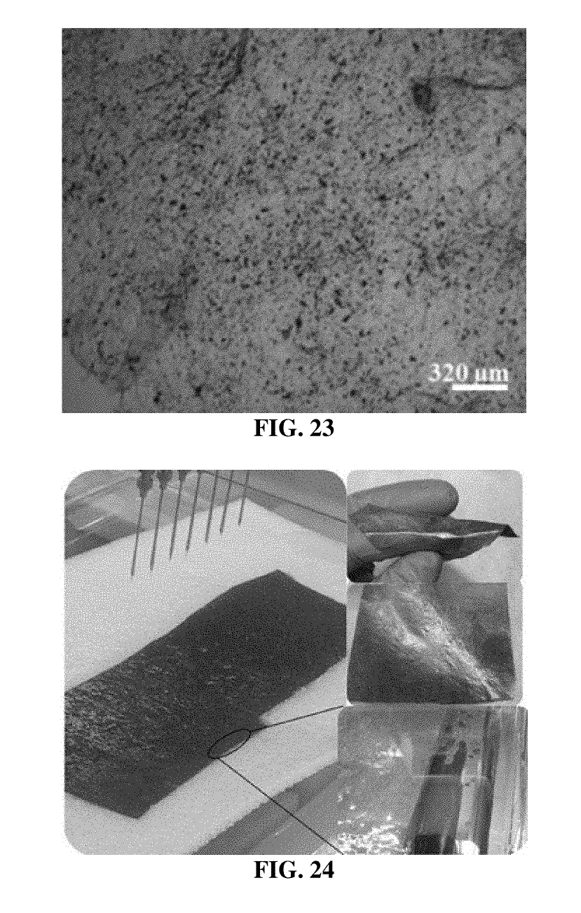

Regenerated chitin (0.4 wt %)/graphene (0.0054 wt %) composite solutions were electrospun to form composite mats. As a source of graphene AO-2 (thickness 8 nm and lateral size ˜550 nm with an overall size distribution of 150-3000 nm) and AO-4 (thickness 60 nm, lateral size ˜3-7 μm) flakes were used for comparison. Electrospinning of the composite regenerated chitin/graphene (AO-2) resulted in strong mat formation on the water surface (FIG. 17 and FIG. 18). The air-dried composite mats were light grey in color, the presence of graphene in the structure was confirmed by PXRD (FIG. 19), and graphene distribution was determined with optical microscopy (FIG. 20-FIG. 23).

Electrospinning of Regenerated Chitin and Shrimp Shell Solutions with AO-2 as a Source of Graphene on a Solid Support

To electrospin composite solution on a solid support, the support was fixed on the surface of water bath to ensure complete wetting of the material. Electrospinning of chitin and composite chitin/graphene solutions was performed according to the procedure described above.

Regenerated chitin (0.4 wt %)/graphene (0.0054 wt %) and shrimp shell solution/graphene (0.0054 wt %) composite solutions were electrospun directly onto solid support to form a support coated with composite chitin/graphene fibers. Electrospinning resulted in complete surface coverage of the support, where the composite fibers were solidified and attached to the support surface after air-drying (FIG. 24).

### Example 2

Chitin/graphene oxide or chitin/graphene composite fibers were produced using a multivariate experimental approach, by varying process variables, including the chitin/graphene ratio, mass loading of biopolymers in the ionic liquid, and spinning conditions. Here chitin was dissolved first in the ionic liquid and graphene was added to the solution prior to spinning or solution was prepared as described above for electrospinning with exception of chitin concentration being 2 wt % in respect to ionic liquid. Ionic liquid solutions of chitin containing suspended graphene particles was used in a dry-jet wet spinning process to prepare graphene or graphene oxide-embedded chitin fibers by coagulation into an aqueous bath. The morphology, physical, and mechanical properties of these fibers as a function of graphene or graphene oxide loading was determined and compared to original fibers with no graphene.

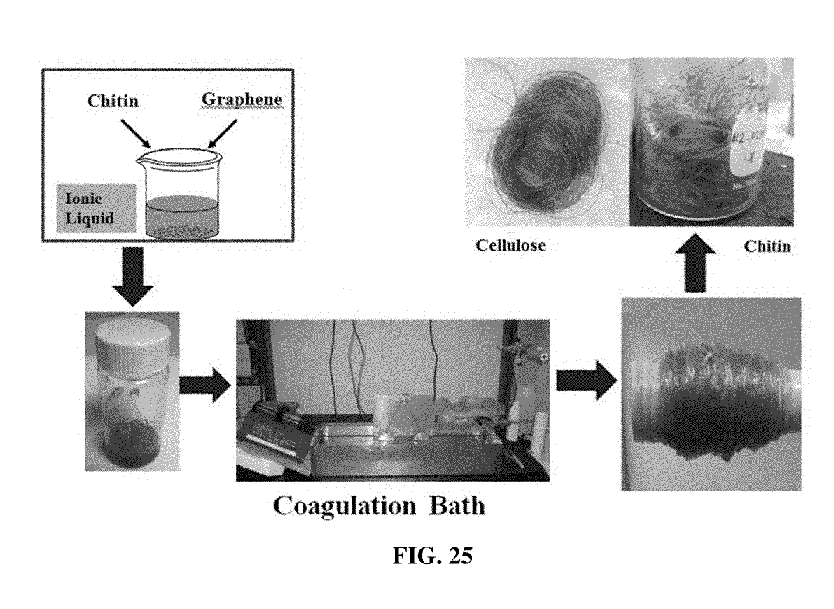

A dry-wet spinning technique was used for the fiber pulling. Fibers were extruded from a syringe with a help of syringe pump. Fibers were pulled through godets submerged in a water or ethanol coagulant. Coagulation occurred by diffusion of the ionic liquid out of the fiber (FIG. 25). These fibers can be further weaved into a textile.

The chitin-graphene oxide (or graphene) fiber pulling process depended on solution viscosity, relative concentration of both chitin and graphene or graphene oxide, and molecular weight of chitin. Cellulose-graphene or graphene oxide fibers were pulled in similar fashion and resulted in fibers that were dark in color.

Cellulose/Graphene or Graphene Oxide Composite Fibers

Cellulose/graphene or graphene oxide composite fibers were produced in similar fashion as chitin/graphene or graphene oxide fibers with the exception that a [C₄mim][Cl] ionic liquid and biopolymer concentration range from 3.75 to 8 wt % were used. Briefly, cellulose was dissolved first in the ionic liquid and then graphene was added to the solution prior to spinning. Ionic liquid solutions of cellulose containing graphene flakes were used in a dry-jet wet spinning process to prepare graphene or graphene oxide-embedded cellulose fibers by coagulation into an aqueous bath (FIG. 25).

Chitin/Graphene Fibers

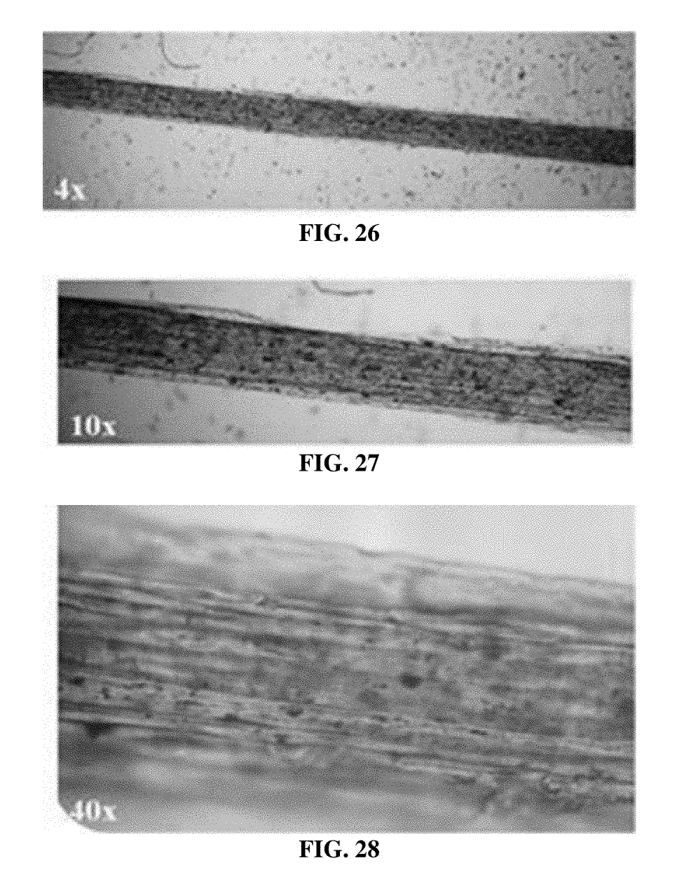

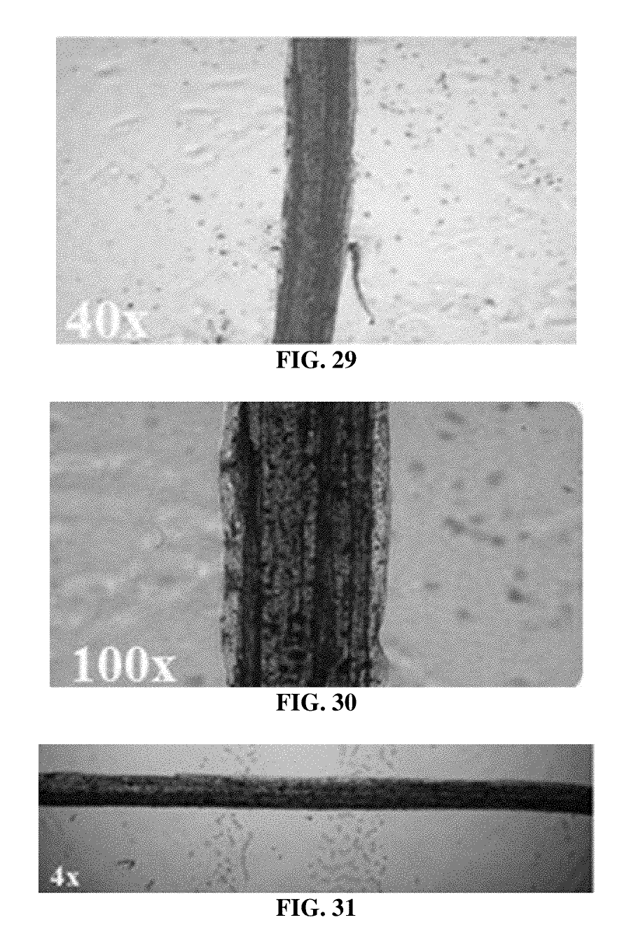

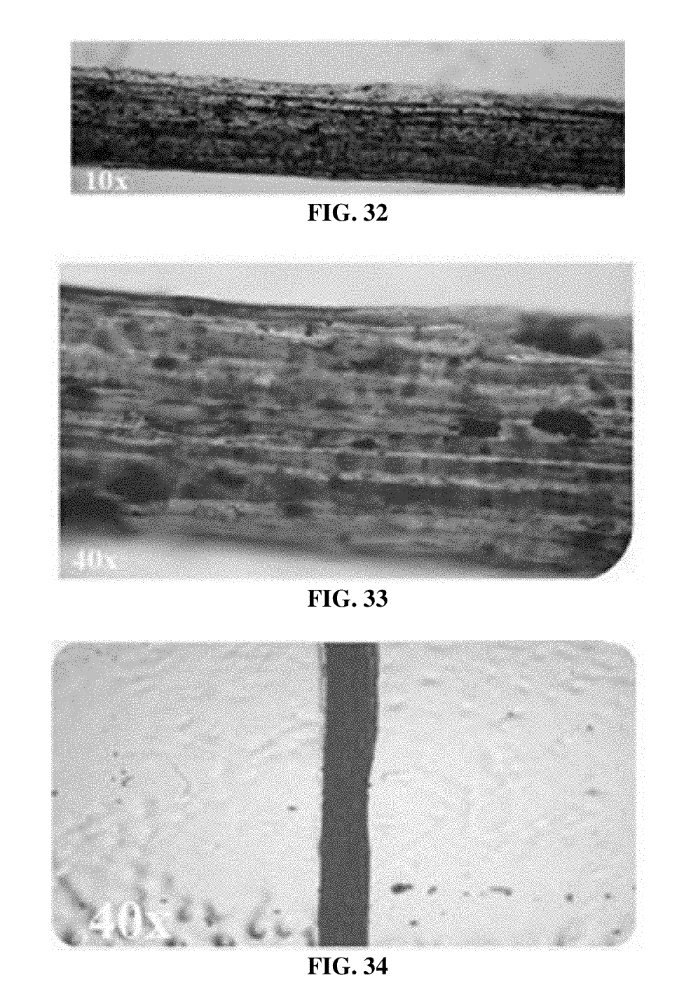

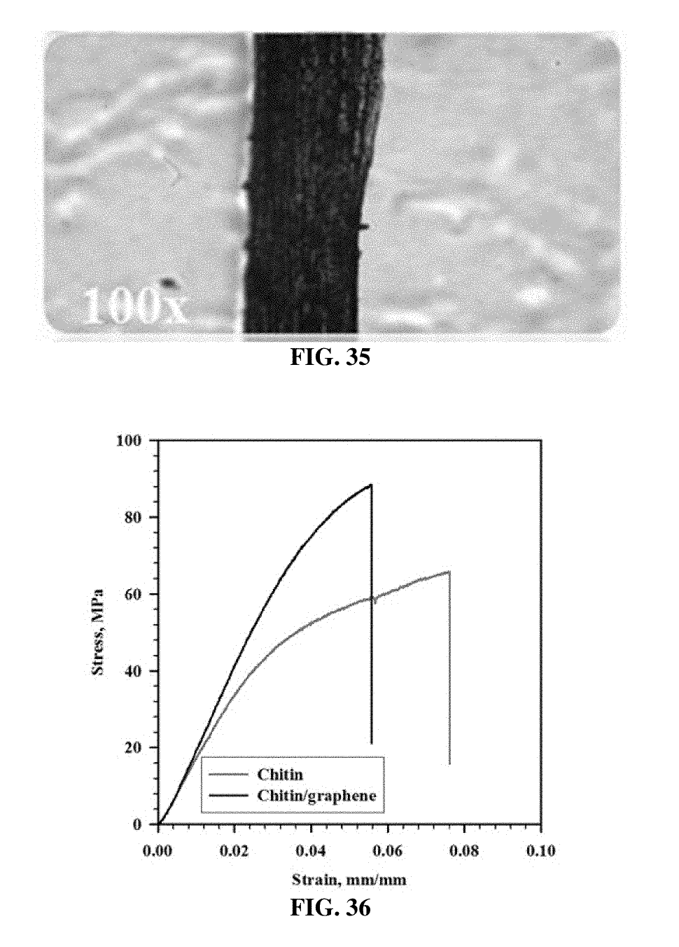

Fibers were pulled from the composite chitin-graphene solutions with graphene concentration of 0.005 wt % and 0.1 wt % and biopolymer concentration of 2 wt %. The fiber morphology and presence of graphene in spun fibers were studied with an optical microscope. As seen from FIG. 26 through FIG. 35, graphene is present in the composites and the packing density of graphene increases with increasing initial graphene loads. The mechanical properties of the graphene-chitin fibers was also investigated. The composite fibers showed increased tensile strength as compared to pure chitin fibers (FIG. 36).

### Example 3

Chitin/Graphene or Graphene Oxide Films

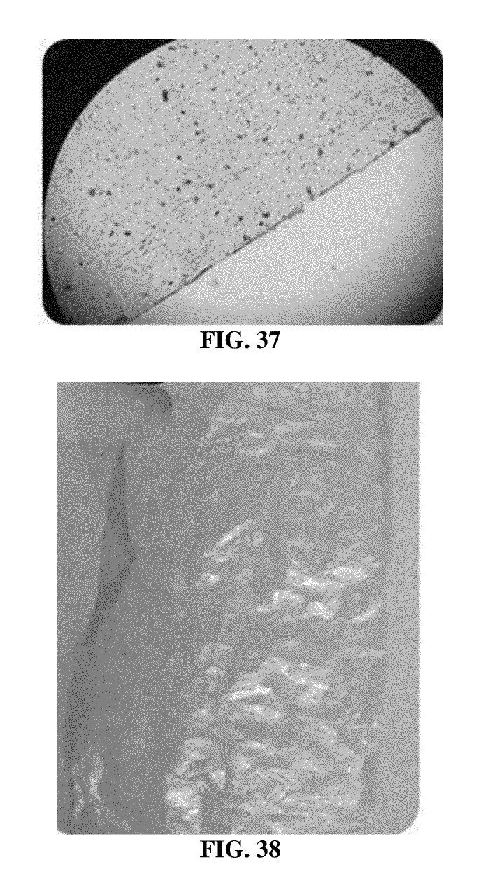

Films were obtained by casting composite chitin-graphene solutions with 0.005 wt % and 0.01 wt % of graphene. The films were rolled with 100 RDS rod and were coagulated in the water bath. The solid films were dried under press in air. The morphology of the casted films were investigated with an optical microscope; the microscopy image clearly shows the presence of well-distributed graphene in the casted film (FIG. 37 and FIG. 38).

### Example 4

All materials were used as supplied unless otherwise noted. The ionic liquid (IL) [C₂mim][OAc], purity >95% was purchased from IoLiTec, Inc. (Tuscaloosa, Ala., USA). Graphene nanopowder, grade AO-3 (specific surface area 80 m²/g, average particle size 4500 nm), was purchased from Graphene Supermarket (Calverton, N.Y., USA).

Commercially sourced graphene was chosen for the preparation of various graphene/chitin composite films. Different ratios of graphene were incorporated in the chitin-ionic liquid solution for dispersion and composite film preparation, as shown in Table 2. A 1.25 wt % solution of chitin was prepared by dissolving 0.0632 g chitin in 4.99 g ionic liquid, and a 1.5 wt % solution of chitin was prepared by dissolving 0.0632 g chitin in 4.15 g ionic liquid. A stir bar was added to each chitin solution, and each solution was placed in an oil bath at 90° C. to dissolve for 18 h for the 1.25 wt % solution and 24 h for the 1.5 wt % solution. Different dissolution times were used for the different chitin solutions loadings, as a higher chitin loading can require a longer time for full dissolution of the chitin. Once the chitin dissolved (monitored visually), AO-3 graphene powder (specific surface area 80 m²/g, average particle size 4500 nm) was added to the solutions in various amounts to prepare samples 1-5 as described in Table 2. For the preparation of graphene slurries in 1.25 wt % chitin/ionic liquid solutions (containing 0.0632 g chitin), 0.0948 g, 0.1474 g, 0.2528 g, and 0.5677 g graphene was added to prepare mixtures of having a ratio of 60:40, 70:30, 80:20, and 90:10 graphene:chitin, respectively, as shown in Table 2.

TABLE 2
Composition of chitin-graphene films.

| | | Ionic | | | |
|---|---|---|---|---|---|
| | | Liquid | | Graphene | |
| Sample | Graphene:Chitin | (g) | Chitin | (g) | Film |
| Sample 1 | 60:40 | 4.99 | 1.25 wt %, | 0.0948 | Successful, dispersion |
| | | | 0.0632 g | | of graphene |
| Sample 2 | 70:30 | 4.99 | 1.25 wt %, | 0.1474 | Successful, dispersion |
| | | | 0.0632 g | | of graphene |
| Sample 3 | 80:20 | 4.99 | 1.25 wt %, | 0.2528 | Successful, dispersion |
| | | | 0.0632 g | | of graphene |
| Sample 4 | 90:10 | 4.99 | 1.25 wt %, | 0.5677 | Successful, dispersion |
| | | | 0.0632 g | | of graphene. Film too |
| | | | | | fragile, to handle |
| | | | | | properly |
| Sample 5 | 80:20 | 4.15 | 1.50 wt %, | 0.2528 | Paste (no dispersion) |
| | | | 0.0632 | | |

The graphene AO-3 powder tended to form aggregates when first added to the chitin solutions, and was dispersed by stirring using a magnetic stir bar for 4 h at 90° C. (e.g., the dispersion period). During the dispersion period, the 1.5 wt % chitin solution formed a paste due to the high solution viscosity and large amount of graphene added, leading to nonhomogeneous mixtures which could not be cast into films (e.g., sample 5, Table 2). The 1.25 wt % chitin/ionic liquid/graphene solution, however, allowed for the complete dispersion of graphene at all graphene loadings, and each remained a free-flowing liquid (e.g., Samples 1-4, Table 2). Once the graphene was well dispersed in the 1.25 wt % chitin solutions (monitored visually), the chitin-graphene films were cast from the solutions according to a previously described method (King et al. Green Chem., 2017, 19, 117-126), with a minor modification in the casting method.

Briefly, solutions of graphene dispersed into chitin/ionic liquid solution were placed into an oven at 90° C. until warm and free-flowing. Films were cast on a glass plate using a double blade micrometer film applicator (MTI Corporation, Richmond, Calif., USA) at a casting height of 75 μm. The casting height of the film applicator will not produce films of that thickness, as the solution tends to spread slowly on the glass and the film swells in the coagulation bath, leading to a wet thickness not equal to that set by the casting knife. The glass plate with the chitin solution mounted was submerged into a DI water bath for coagulation. The water was replaced 4-5 times, with about 20 min in between, to remove all of the ionic liquid. The wet films were inspected for strength and homogeneity, and the films of 60, 70, and 80 wt % graphene were homogenous and free of visible defects or tears. However, the 90 wt % graphene film was very fragile, with small pieces of the film coming apart. The films were then removed from the coagulation bath and press dried between two pieces of parchment paper under a flat weight (ca. 2 kg) overnight. The steps of dissolution, casting, coagulation, and press drying were performed in the same manner as for neat chitin films. For neat chitin films, 1.25 wt % chitin ionic liquid solutions were cast, coagulated, and press dried in the same manner as for the composite graphene/chitin films.

Upon drying, the 60, 70, and 80 wt % films all remained in one piece and were completely black and opaque. These films were flexible and could be manipulated, bent, and cut. The 90 wt % graphene film, however, broke into small pieces upon drying and could not be used. This is likely due to insufficient interactions between polymer chains due to the high loading of graphene and because of this, the 90 wt % films were not further studied.

Highest loading film, 80 wt % graphene, was selected for further characterization. All further discussion of the graphene/chitin film will refer explicitly to the 80 wt % graphene/chitin composite films.

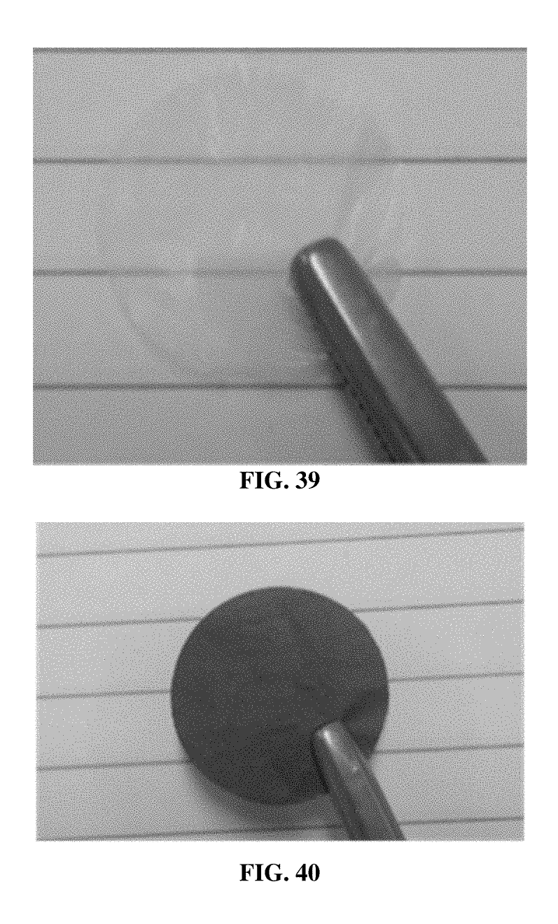

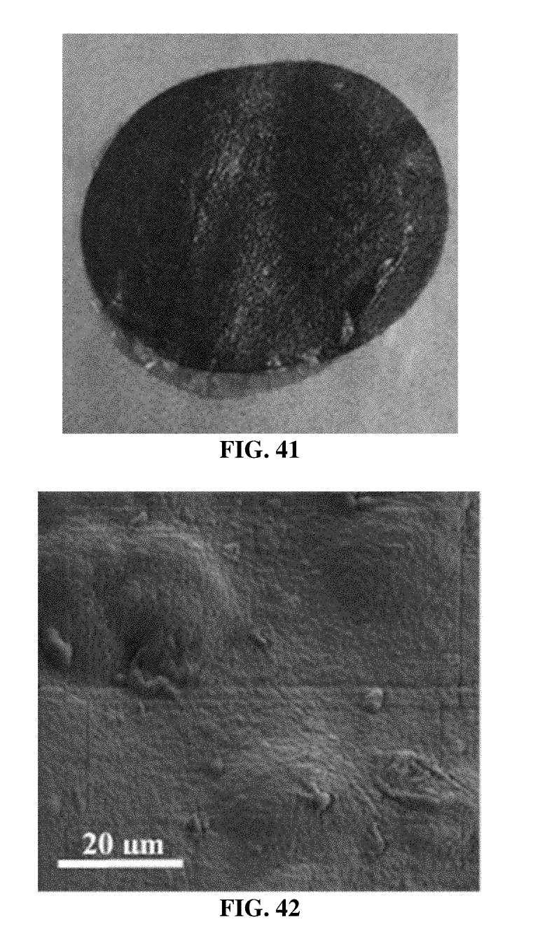

Photographs of the 80 wt % graphene/chitin composite film and a neat chitin films are shown in FIG. 39-FIG. 41. Table 3 summarizes certain properties of the neat chitin film and 80 wt % graphene/chitin composite film, which will be discussed further below.

TABLE 3
Observations and properties of neat chitin film and
80 wt % graphene/chitin composite films.

| | | | 80 wt % graphene/ |
|---|---|---|---|
| Property | | Neat Chitin Film | chitin composite film |
| Observations | | Film is translucent and | Film is completely |
| | | flexible. The thin film | black, and appears |
| | | could be manipulated | homogenous. The thin |
| | | and bent with ease. | film could be |
| | | | manipulated, but was |
| | | | brittle. |
| Thickness (mm) | | 0.026 | 0.079 |
| Thermal Stability | | 266 | 246 |
| T_{5%dec} (° C.) | | | |
| Mechanical | Tensile | 5 (1) | 1.7 (2) |
| | Strength | | |
| Properties | Young's | 704 (46) | 257 (70) |
| (MPa) | Modulus | | |

| | | |
|---|---|---|
| Swelling ratio in | 333 | 220 |
| electrolyte (%) | | |

The surface morphologies of both neat and composite films and the homogeneity of graphene dispersed in the electrode film were inspected using a Delong America LVEMS 5 kV benchtop scanning electron microscope electron microscope (Montreal, QC, CA). Scans were taken using a 5 kV electron beam.

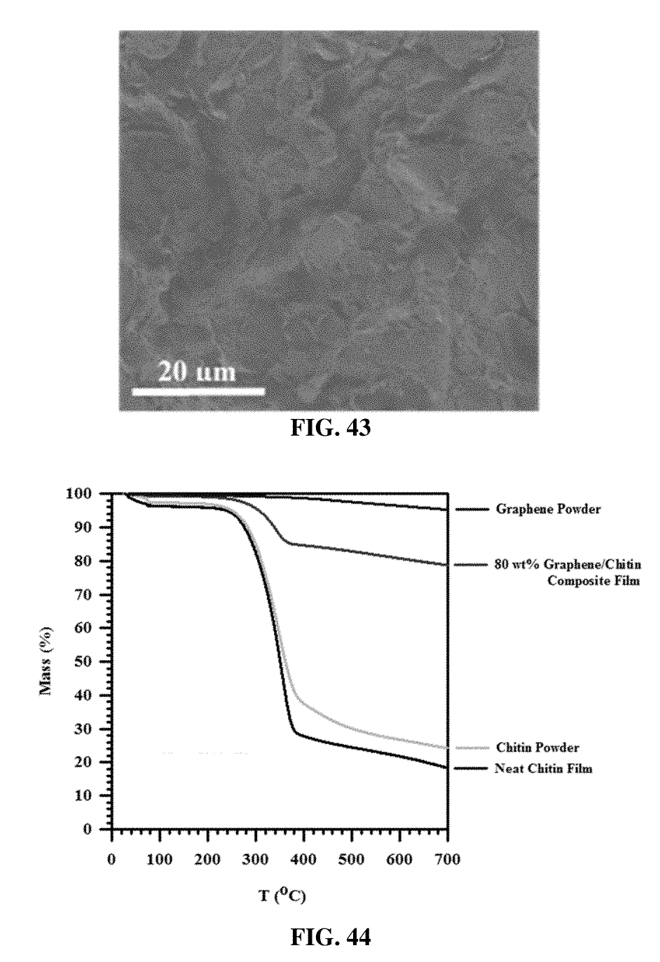

The SEM images of the neat chitin film, shown in FIG. 42, revealed an uneven and nonporous surface. Images of the graphene/chitin composite films, shown in FIG. 43, revealed a homogeneous distribution of graphene down to the micrometer scale, with no pores, breakage, or cracks observed, demonstrating the successful incorporation of graphene into the chitin film.

For a better understanding of the physical properties of the films and of the interactions between the two components in the composite, thermal and mechanical properties of the film were measured by thermogravimetric analysis (TGA) and tensile testing, respectively.

Thermogravimetric analysis was performed on a TA Instruments Q500 TGA instrument (New Castle, Del., USA), with initial heating from room temperature to 75° C., then holding with a 30 min isotherm, followed by heating to 700° C. using a heating rate of 5° C./min Samples of 2-5 mg were analyzed in 70 μL alumina pans. Decomposition temperatures are reported at 5 wt % mass loss (T_{5% dec}).

Because the graphene does not decompose until very high temperatures, the TGA curve shown for the composite film in FIG. 44 has been normalized to the mass of chitin in the film in order to better compare to the neat chitin film. The decomposition temperature of 5 wt % of the material (T_{5% dec}) and the overall mass loss profile were found to be similar for the neat chitin film and the graphene/chitin composite films, with values of 266° C. and 246° C., respectively. (These are similar to the value of 253° C. found for neat chitin films previously reported.) This indicates that the decomposition of the composite material is due to the decomposition of chitin alone, and the preparation of the composite film does not affect the thermal stability of the chitin itself. This also suggests that the interactions between the chitin and graphene are weak, and that the composite is formed through physical interactions rather than chemical interactions between the two film components. The sharp decrease in mass in the composite film TGA after 400° C. is an artifact of the normalization, as the graphene in the material is also losing mass (FIG. 44 for TGA of neat graphene, both films, and neat chitin).

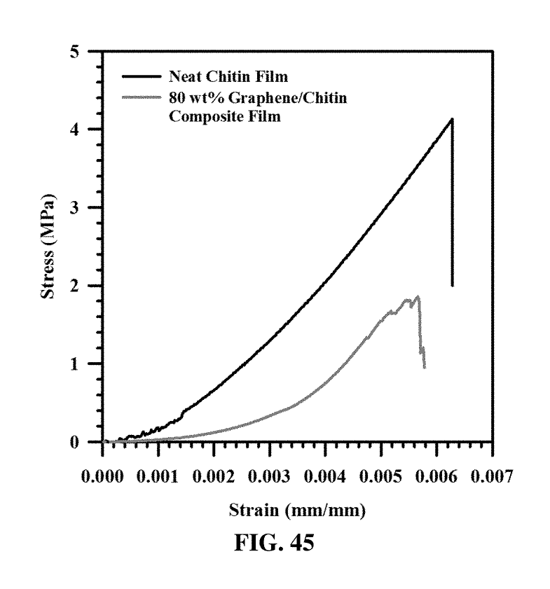

Tensile testing of films was conducted using a Test Resources 220Q Universal Test Machine (Shakopee, Minn., USA) Films with no obvious flaws were selected and cut into strips of 2 cm wide and 7-10 cm long. Thickness of the films was measured using a micrometer. Stress/strain curves were obtained and reported for each film (FIG. 45). Films were cut into thin strips (2 cm×5 cm) for testing. The tensile strength of neat chitin films was 5(1) MPa, with a Young's modulus of 704(46) MPa. The graphene/chitin composite films had even lower tensile strength, 1.7(2) MPa with a Young's modulus of 257(70) MPa. The lowering of the strength is due to incorporation of the graphene, which does not have good adhesion to the chitin, and lessens the interactions between the chains of the biopolymer, lowering the strength of the material.

The methods and compositions of the appended claims are not limited in scope by the specific methods and compositions described herein, which are intended as illustrations of a few aspects of the claims and any methods and compositions that are functionally equivalent are within the scope of this disclosure. Various modifications of the methods and compositions in addition to those shown and described herein are intended to fall within the scope of the appended claims. Further, while only certain representative methods, compositions, and aspects of these methods and compositions are specifically described, other methods and compositions and combinations of various features of the methods and compositions are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

What is claimed is:

1. A method of making a graphene-biopolymer composite material comprising:
contacting an ionic liquid with a biopolymer and graphene, thereby forming a mixture;
contacting the mixture with a non-solvent, thereby forming the graphene-biopolymer composite material in the non-solvent; and
collecting the graphene-biopolymer composite material from the non-solvent.

2. The method of claim 1, wherein the ionic liquid comprises a cation and an anion, wherein the cation is selected from the group consisting of: 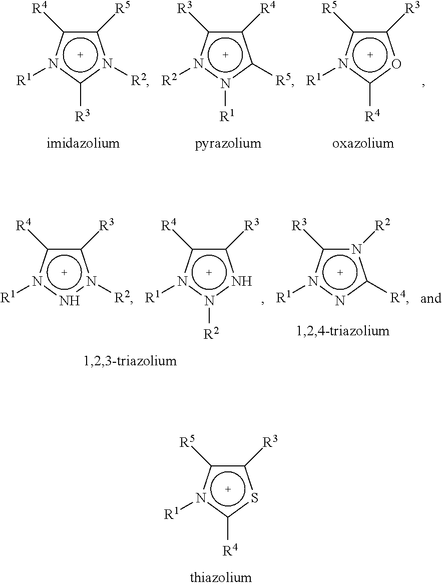
where each R¹ and R² is, independently, a substituted or unsubstituted linear, branched, or cyclic C₁-C₆ alkyl, or substituted or unsubstituted linear, branched, or cyclic C₁-C₆ alkoxy; each R³, R⁴, and R⁵ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl; and
wherein the anion is selected from the group consisting of C₁₋₆ carboxylate, halide, CO₃²; NO₂⁻, NO₃⁻, SO₄²⁻, CN⁻, R¹⁰CO₂, (R¹⁰O)₂P(═O)O, (R¹⁰O)S(═O)₂O, or (R¹⁰O)C(═O)O; where R¹⁰ is hydrogen; substituted or unsubstituted linear, branched, or cyclic alkyl; substituted or unsubstituted linear, branched, or cyclic alkoxy; substituted or unsubstituted aryl; substituted or unsubstituted aryloxy; substituted or unsubstituted heterocyclic; and substituted or unsubstituted heteroaryl.

3. The method of claim 1, wherein the ionic liquid contains an imidazolium cation.

4. The method of claim 1, wherein the ionic liquid is a 1-alkyl-3-alkyl imidazolium C₁-C₆ carboxylate or a 1-alkyl-3-alkyl imidazolium C₁-C₆ carboxylate halide.

5. The method of claim 1, wherein the ionic liquid is 1-ethyl-3-methyl-imidazolium acetate ([C₂mim]OAc), or 1-butyl-3-methyl-imidazolium chloride ([C₄mim]Cl).

6. The method of claim 1, wherein the concentration of biopolymer in the mixture is from 0.1 wt % to 30 wt % with respect to the weight of the ionic liquid.

7. The method of claim 1, wherein the biopolymer comprises chitin, chitosan, cellulose, hemicelluloses, or a combination thereof.

8. The method of claim 1, wherein contacting the ionic liquid with the biopolymer comprises dissolving or dispersing at least a portion of a source of the biopolymer in the ionic liquid.

9. The method of claim 1, wherein the concentration of graphene in the mixture is from 0.01 to 90 wt % compared to the amount of biopolymer in the mixture.

10. The method of claim 1, wherein the graphene comprises flakes of graphene with an average maximum lateral dimension of 1 nm to 100 μm.

11. The method of claim 1, wherein contacting the ionic liquid with the biopolymer and graphene comprises:
contacting the ionic liquid with the graphene to form a precursor mixture and contacting the precursor mixture with the biopolymer to form the mixture;
contacting the ionic liquid with the biopolymer to form a precursor mixture and contacting the precursor mixture with the graphene to form the mixture;
or
contacting the ionic liquid with the biopolymer to form a first precursor mixture, contacting the ionic liquid with the graphene to form a second precursor mixture, and contacting the first precursor mixture with the second precursor mixture to form the mixture.

12. The method of claim 1, wherein the ionic liquid is contacted with the graphene and biopolymer under agitation.

13. The method of claim 1, further comprising agitating the mixture and/or heating the mixture at a temperature of from 25° C. to 190° C.

14. The method of claim 1, wherein the non-solvent is water, a C₁-C₄ alcohol, ketone, or a mixture thereof.

15. The method of claim 1, wherein contacting the mixture with non-solvent comprises contacting the mixture with a substrate submerged in the non-solvent, thereby coating the substrate with the composite graphene-biopolymer material.

16. The method of claim 1, wherein the graphene is substantially homogeneously dispersed throughout the graphene-biopolymer composite material.

17. The method of claim 1, further comprising separating at least a portion of the ionic liquid from the non-solvent, thereby forming a recycled ionic liquid, and wherein the recycled ionic liquid is used to contact the biopolymer and graphene.

18. The method of claim 1, wherein the graphene-biopolymer composite material is formed into a fiber, a film, a bead, a mat, or a combination thereof.

19. A composition comprising the graphene-biopolymer composite material made by the method of claim 1.

20. An article of manufacture comprising the graphene-biopolymer composite material made by the method of claim 1.
